(19) United States
(12) Patent Application Publication
(10) Pub. No.: US 2015/0111275 A1
(43) Pub. Date: Apr. 23, 2015

(54) OPTICAL REGULATION OF GENE EXPRESSION IN THE RETINA

(71) Applicants: Daniel V. Palanker, Sunnyvale, CA (US); Thomas W. Chalbert, JR., Redwood City, CA (US)

(72) Inventors: Daniel V. Palanker, Sunnyvale, CA (US); Thomas W. Chalbert, JR., Redwood City, CA (US)

(21) Appl. No.: 14/407,054

(22) PCT Filed: Jun. 10, 2013
(86) PCT No.: PCT/US13/45043

§ 371 Date: Dec. 10, 2014

Related U.S. Application Data
(60) Provisional application No. 61/658,316, filed on Jun. 11, 2012.

(51) Int. Cl. A61K 48/00; A61K 41/00; A61F 9/008; C12N 15/86

(52) U.S. Cl. CPC A61K 48/005; C12N 15/86; A61K 41/0057; A61F 9/008; C12N 2750/14143; C12N 2830/002; A61F 2009/00863; A61F 2009/00876; A61F 2009/00868
USPC 435/173.8; 606/10; 606/4; 606/11

(57) ABSTRACT

The present invention provides methods and compositions for regulating transgene expression in a subject using heat generated by electro-magnetic radiation in a target cell. The heat is generated by a thermo generator resided in the target cell upon application the electromagnetic radiation. The amount of the heat is controlled by the energy delivered by the electromagnetic radiation.

This application is a U.S. national phase of PCT/US2013/045043, filed Jun. 10, 2013, which application claims priority under 35 USC §119(e) to U.S. Provisional Application No. 61/658,316, filed Jun. 11, 2012, each of which is incorporated herein by reference in its entirety.

### BACKGROUND OF THE INVENTION

Gene therapy generally refers to the use of DNA as a pharmaceutical to treat disease. In general, gene therapy involves replacing a defective gene with a functional copy of that gene. Other forms include directly correcting a mutation, or using DNA that encodes a therapeutic protein or RNA, which may or may not be naturally occurring, but does not necessarily replace a defective copy in an individual, to provide treatment. In gene therapy applications, DNA is generally packaged in a “vector” that provides functional delivery of the therapeutic DNA to target cells in the body.

Among gene therapy vectors in clinical use, some vectors are able to replicate and persist within dividing cells, including vectors that stably integrate into the genome, such as retrovirus, lentivirus, phiC31 integrase, and Sleeping Beauty, piggyBAC, and other transposases, as well as vectors that can replicate without genomic integration, such as the Epstein-Barr virus system. Other gene therapy vectors are not able to persist within dividing cells, and are therefore “lost” during cell division. This class includes viral vectors such as adenovirus, AAV, herpes virus, and plasmid DNA lacking eukaryotic replication mechanisms (whether or not complexed with agents to facilitate delivery).

Depending on the vector used and the particular clinical use, the therapeutic DNA may persist in tissues for an extended period following initial delivery, and in some instances, will continue to express the “transgene” (i.e., make the therapeutic RNA or protein) over a long period. In some clinical applications, such long-term, persistent expression may be undesirable or have the potential to cause concern, for example, if there is a complete resolution of the disease and continued expression is unnecessary, or if transgene expression leads to adverse events, such as a hypersensitivity reaction or acute or chronic toxicity. In such cases, continued expression of the transgene could be undesirable or problematic, and it would be desirable to “turn off” or reduce transgene expression, or otherwise “remove” cells containing the transgene. In other cases, the level of transgene expression is too low resulting in a subtherapeutic dose of the transgene product. It would be desirable to increase, at least temporarily, or permanently, the level of trans gene expression. Even more desirable would be the capacity to tune, or adjust the level of trans gene expression, up or down in a stepwise fashion so as to more finely control the ultimate levels.

To address this long-standing need in the art, several systems have been designed to regulate transgene expression following gene therapy. Many such systems use a small-molecule-inducible system, where a transcription factor includes a regulatory domain that, upon binding of a small molecule, changes the conformation of the transcription factor to make it functionally active. In these systems, continued presence of the small molecule drug is required for gene regulation. For example, the presence of tetracycline or doxycycline keeps transgene expression on (in the Tet-On system) or off (e.g., in the Tet-Off system). Another such system involves a dimerizer-regulated system, where a chimeric activation domain is fused to the rapamycin-binding domain of FRAP, and rapamycin is used to up-regulate gene expression levels. Other systems use steroid hormone analogs, such as the progesterone receptor regulatory system. In this system, mifepreistone (RU 486) binds a C-terminal truncated progesterone receptor, in some cases fused to DNA binding and activation domains. Another system uses the ecdysone receptor (EcR) from Drosophila melanogaster. Still other systems have used demonstrated transgene upregulation driven by hypoxia responsive elements, nutrient-deficiency, or ionizing X-ray radiation.

Despite great efforts placed on development of inducible gene expression systems, the developed approaches have been cumbersome and provide an incomplete solution in most clinical settings. The most widely used systems, such as Tet-On, Tet-Off, rapamycin-induced dimerization, and steroid-hormone regulation, require that a drug be taken on a constant basis to maintain expression in the “on” or “off” state. The small molecule agent used for induction may itself have undesirable effects, such as off-target biological effects of rapamycin (e.g., immunosuppression), and off-target effects of steroid hormones, which would be similar to hormone-replacement therapy effects at the levels proposed for the RU-486 system. Systems that seem to avoid this problem, such as the TetOn system, suffer from other shortcomings that prevent widespread clinical use, such as immune response to the gene product in large animals. Another shortcoming is that these systems are often “leaky”, leading to a low level of background expression persisting in the “off” state. Finally, for such a system to reach widespread clinical use, substantial barriers would need to be overcome in the clinical trial design phase of drug development. Such an approach would necessitate independent arms in a clinical trial to demonstrate safety of the gene therapy agent alone, the inducible agent alone, plus the combination, and in the later phases employ a randomization scheme to switch off the therapy. Randomizing to switch off a therapy that may provide benefit poses an ethical challenge, and using the switch only “when needed” is problematic analytically.

### SUMMARY OF THE INVENTION

The present invention provides compositions and methods for modulating gene expression in a subject.

In one aspect, the present invention provides a method for modulating gene expression in a subject, comprising:(a) expressing a transgene in a plurality of cells in a subject, wherein the plurality of cells comprises a target cell, wherein the target cell comprises: (i) a polynucleotide encoding said transgene; and (ii) a chromophore that is capable of generating heat upon application of an electromagnetic radiation; and (b) applying the electromagnetic radiation to the target cell for a suitable period of time to generate heat to damage or destroy the target cell, thereby modulating expression of the transgene in the subject.

In some aspects, the target cell is a pigmented cell and said chromophore comprises a pigment present in a cell type selected from the group consisting of: retinal pigment epithelium, iris epithelium, trabecular meshwork epithelium, and a cell in the pigmented choroid. The chromophore can include melanin.

In some aspects, the transgene is an anti-angiogenic factor, selected from the group consisting of: sFlt-1, sFLT01, VEGF trap (also known as aflibercept), an anti-VEGF antibody or antibody fragment, an anti-VEGF soluble receptor or receptor fragment, an Fc fusion protein comprising a soluble FLT peptide or fragment, soluble FLT peptides or fragments, PEDF, angiostatin, endostatin, TIMP3, or a PDGF inhibitor. In some aspects, the transgene is a neurotrophic or anti-apoptotic factor, selected from the group consisting of:. GDNF, CNTF, BDNF, NTN, NT-4, NGF, or RdCVF, PDGF-R. In some aspects, the transgene modulates (either increase or decrease) a genetic disease, and can be selected from the group consisting of: RPE65, retinoschisin, CRB1, retinitis pigmentosa GTPase regulator (RGPR)-interacting protein-1, peripherin, peripherin-2 (Prph2), a light-responsive opsin (ChR2; Chop2, L-opsin (OPN1LW), M-opsin (OPN1MW), S-Opsin (OPN1SW), rhodopsin (Rh1, OPN2, RHO), fibroblast growth factor 2, nurturin, epidermal growth factor, or complement inhibitor.

In some aspects, the transgene is carried by a vector, such as a virus or a plasmid. In some aspects, the transgene is carried by a replicative vector, such as a retrovirus, lentivirus, phiC31 integrase, Sleeping Beauty, piggyBAC or other transposases. In some aspects, the transgene is carried by a non-replicative vector, such as a recombinant AAV in a wildtype, mutated, or not naturally occurring form.

In some aspects, the electromagnetic radiation is produced by laser. In some aspects, the laser has a beam width of less than 1 mm. In some aspects, the laser power has an intensity of about 500 mW to 5,000 mW. In some aspects, the electromagnetic radiation is applied in pulse, such as the duration of the pulse of electromagnetic radiation is less than 100 μs.

In some aspects, the electromagnetic radiation is applied by scanning a beam of electromagnetic radiation. In some aspects, electromagnetic radiation may be applied through beam scanning. In some aspects, the scanning velocity of said beam is about 1, 2, 3, 4, 5, 6, 7, 8, 9, 10 m/s or higher. In some aspects, the scanning beam has a dwell time less than 100 μs.

In another aspect, the present invention provides a method for modulating gene expression in a target cell by electromagnetic radiation, comprising: (a) expressing a transgene in a target cell, wherein the target cell comprises: (i) a polynucleotide encoding a heat-responsive promoter that controls expression of the transgene; and (iii) a chromophore that is capable of generating heat upon application of an electromagnetic radiation; and (b) applying said electromagnetic radiation to the target cell for a suitable period of time to increase the expression of said transgene.

In some aspects, the target cell is a pigmented cell and can be selected from the group consisting of: retinal pigment epithelium, iris epithelium, trabecular meshwork epithelium, and a cell in the pigmented choroid. The chromophore can include melanin.

In some aspects, the transgene is an anti-angiogenic factor, selected from the group consisting of: sFlt-1, sFLT01, VEGF trap (also known as aflibercept), an anti-VEGF antibody or antibody fragment, an anti-VEGF soluble receptor or receptor fragment, an Fc fusion protein comprising a soluble FLT peptide or fragment, soluble FLT peptides or fragments, PEDF, angiostatin, endostatin, TIMP3, or PDGF inhibitor. In some aspects, the transgene is a neurotrophic or anti-apoptotic factor, selected from the group consisting of:. GDNF, CNTF, BDNF, NTN, NT-4, NGF, or RdCVF, PDGF-R. In some aspects, the transgene modulates a genetic disease, and can be selected from the group consisting of: RPE65, retinoschisin, CRB1, retinitis pigmentosa GTPase regulator (RGPR)-interacting protein-1, peripherin, peripherin-2 (Prph2), a light-responsive opsin (ChR2; Chop2, L-opsin (OPN1LW), M-opsin (OPN1MW), S-Opsin (OPN1SW), rhodopsin (Rh1, OPN2, RHO), fibroblast growth factor 2, nurturin, epidermal growth factor or complement inhibitor.

In some aspects, the vector is non-replicative, such as a recombinant AAV in a wildtype, mutated, or not naturally occurring form. In other aspects, the vector is an integrating or replicative vector.

In some aspects, the electromagnetic radiation is light emitted by laser having a beam width in the range from 10 to 1000 μm. In some aspects, the laser has a power of about 5 mW to 5000 mW.

In some aspects, the electromagnetic radiation is applied in pulse, with duration of less than 100 μs. Pulse radiant exposure can be in the range of from about 100 to 1000 mJ/cm².

In some aspects, the electromagnetic radiation is applied by scanning a beam of electromagnetic radiation. In some aspects, electromagnetic radiation may be applied by beam scanning. In some aspects, the scanning velocity of said beam is about 1, 2, 3, 4, 5, 6, 7, 8, 9, 10 m/s or higher. In some aspects, the scanning beam has a dwell time less than 100 μs.

In some aspects, the power of the laser is lower than a cellular damage threshold.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative aspects, in which the principles of the invention are utilized, and the accompanying drawings of which:

FIG. 1 shows a view of the fundus with a diagram of 3 lines scanned sequentially by a single laser spot.

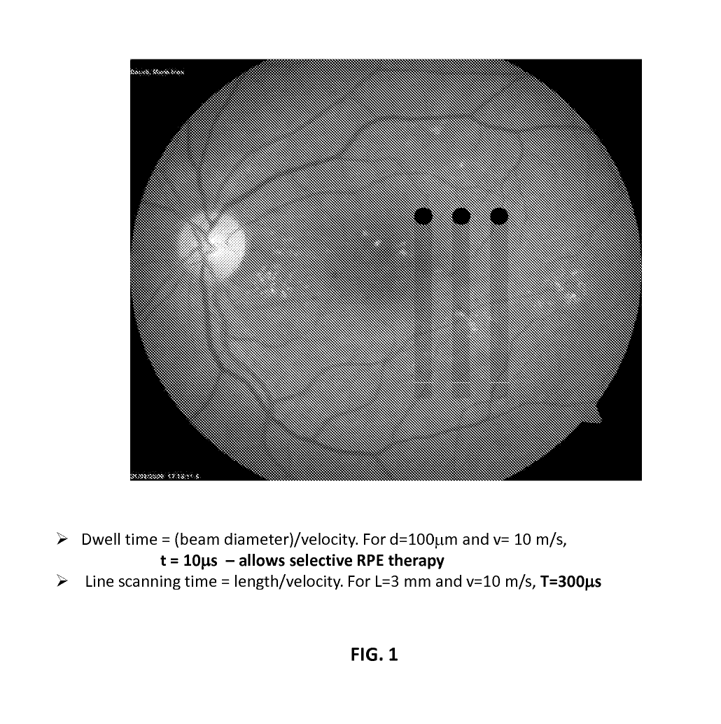

FIG. 2 depicts light microscopy (LM) of selective retina pigmented endothelium (RPE) therapy (SRT) 1 hour after treatment, showing collapse of RPE cells with limited outer segments damage (a). Scanning electron microscopy (SEM) shows absence of RPE cells at the site of SRT with surrounding large RPE cells with foot processes (arrow) after 1 day (b), and transmission electron microscopy (TEM) shows selective ablation of RPE cell with disruption of cytoplasmic membrane, intracellular organelles and basal infolding. However, Bruch's membrane and choroid remains intact (c). After 3 days, LM shows reestablishment of RPE cell layer with no damage to outer nuclear layer (d), migration of surrounding RPE cells on SEM (e) and recovery of tight junctions between RPE cells and presence of normal basal infoldings on TEM (f). After seven days, normal retina morphology is shown in LM (g), with complete restoration of RPE monolayer with presence of large and small RPE cells with microvilli on SEM (h). BrdU uptake assay shows positive RPE cell after 4 days of SRT (i).

FIG. 3 depicts confocal scanning laser ophthalmoscopy fluorescence imaging of an area of the retina that received a subretinal injection of balanced saline solution (BSS) after two months (a), showing normal spectral domain OCT (b) and normal background autoflorescence and DAPI uptake on fluorescence microscopy (FM) (c). Fluorescent imaging of an area of retina 2 months following subretinal injection of 1×10¹¹ vg GFP transgene expression shows uniform expression (d), with normal morphology on SD-OCT (e) and uniform GFP transgene expression evident in the RPE cell layer, with no significant increase of GFP expression in the outer or inner retina on FM (f). rAAV-GFP injected bleb following line-scanning SRT treatment 3 weeks after transfection, showing clear lines of decreased fluorescence (g), with focal RPE increased reflectivity on SD-OCT, but no outer retina damage or disruption of inner/outer segment junction (h), which was confirmed on FM which shows interposed areas of increased and decreased GFP expression in RPE with no increased expression on neurosensory retina (i).

FIG. 4 depicts fluorescence signal brightness of transfected blebs over time. Dashed lines show average brightness over period. Arrows represent when laser therapy (Line scanning SRT) was applied, a.u.: arbitrary units.

FIG. 5 depicts ratio of the GFP expression in Lasered and Control Blebs.

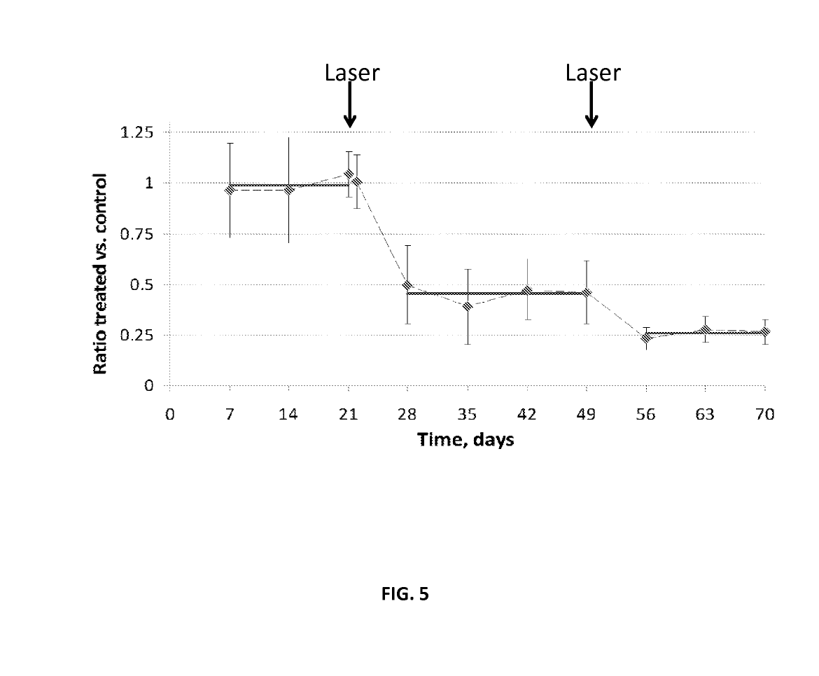

FIG. 6 depicts retreatment with SRT.

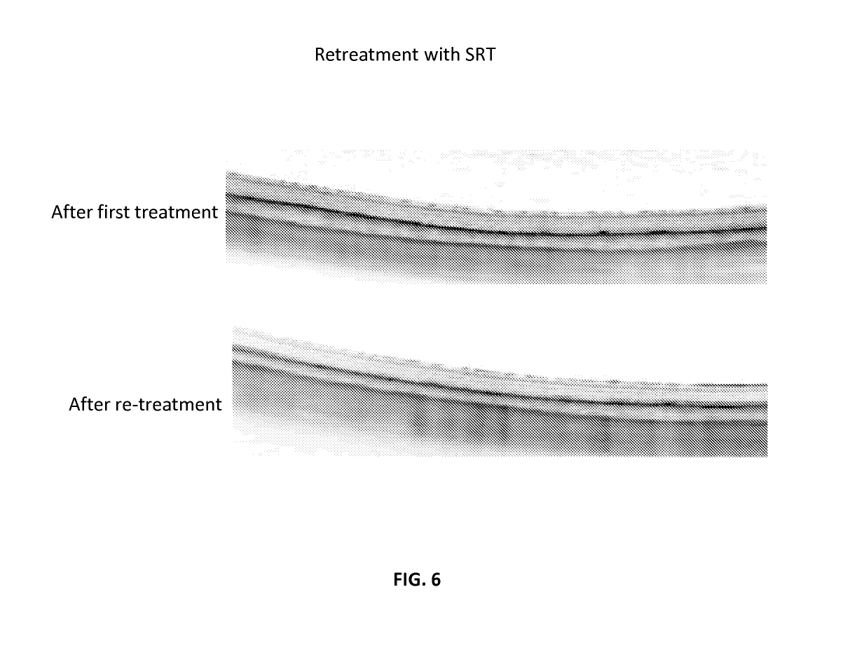

FIG. 7A depicts bioluminescence in the in vivo albino mouse. To visualize luciferase expression, a transgenic mouse was used where luciferase is under the control of a heat-shock sensitive promoter (HSP70). The left eye (control) shows a low-level luciferase expression. The right eye shows elevated luciferase expression following the application of sublethal laser (SLL).

FIG. 7B, further shows that luciferase expression was elevated with increasing power of the SLL applied, where 170 mW>125 mW>95 mW>70 mW.

FIG. 8 depicts elevated expression of luciferase linked to Heat Shock Protein HSP70 following SLL as the bioluminescence ratio between treated and control in the pigmented mouse. Transgene expression increases with increasing SLL power, up to the threshold of RPE damage (40 mW).

### DETAILED DESCRIPTION OF THE INVENTION

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

The terminology used herein is for the purpose of describing particular aspects only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

### I. Overview

The instant invention provided methods and compositions for using electromagnetic radiation provided by a source such as a laser to modulate gene expression in target cells. In some aspects this can be accomplished while avoiding collateral damage to the adjacent layer of cells.

Various technologies have been developed for introduction and control of transgene expression in a host human subject for gene therapy purposes. However, there are many shortcomings in these delivery systems. To address the shortcomings of currently available technology, the present invention provides methods and compositions for regulating transgene expression in a subject using heat generated by electromagnetic radiation. The heat is generated by a thermo generator (e.g. a pigment) residing in the target cell upon application of electromagnetic radiation (e.g. laser). The amount of heat is controlled by the energy delivered by the electromagnetic radiation.

The present invention provides a desirable system for regulation in clinical gene therapy applications that: (a) does not require constant presence or absence of an inducer, (b) utilizes a device, rather than a drug, to facilitate clinical development and adoption, (c) exerts effects locally at the site of transduction, with minimal systemic or off-target effects, and (d) is “tunable” to allow for gradual tuning up or down of transgene expression by a desired amount.

There are at least two possible modes that can be used to regulate gene expression in a target cell by controlling the amount of the heat generated inside the target cell. In the first mode, if sufficient heat is generated, the target cell is damaged, killed, destroyed, or vaporized, which partially reduce, but may also completely eliminates, the transgene expression in the target cell. Alternatively, a suitable amount of heat is generated, which may not be sufficient to destruct or damage the target cell, but is sufficient to modulate the expression of a transgene that is under the control of a heat sensitive promoter, such as a heat shock protein promoter or CMV promoter (Pshenichkin, S. et al. Heat shock enhances CMV-IE promoter-driven metabotropic glutamate receptor expression and toxicity in transfected cells. Neuropharmacology 60, 1292-1300 (2011)). This either directly controls the expression of the target gene, or indirectly controls a second target gene that is downstream of the first target gene (such as a heat-shock protein) in gene regulation pathway.

Both forms of laser-mediated regulation of gene expression can be applied to cells that can be heated by the laser. For selective destruction, target cells should be more amenable to heating compared to surrounding cells/tissues, for example if they contain intracellular chromophores, such as melanosomes.

### II. Modulation of Gene Expression Through Selective Destruction of Target Cells

In one aspect, the present invention provides a method for modulating (either increasing or decreasing) gene expression in a subject through selective damage or destruction of target cells.

The target cells can be selectively damaged or destructed by various methods, either chemically, physically, or biologically. In some aspects, the target cells are damaged or destructed physically, such as with electromagnetic radiation or ultrasound.

In some aspects, the target cells are damaged or destructed by electromagnetic radiation, and the method comprises: (a) expressing a transgene in a plurality of cells in a subject, wherein the plurality of cells comprises a target cell, wherein the target cell comprises: (i) a polynucleotide encoding the transgene; and (ii) a thermo generator that is capable of generating heat upon application of an electromagnetic radiation; and (b) applying the electromagnetic radiation to the target cell for a suitable period of time to generate heat to destruct the target cell, thereby modulating expression of the transgene in the subject.

By “subject” or “individual” herein is meant individuals suffering from a disorder, and the like, encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In some aspects of the methods and compositions provided herein, the mammal is a human.

### A. Target Cells

By “target cell” herein is meant a cell of interest. A target cell generally comprises a transgene (“target cell transgene”) controlled by a regulatory element, such as a promoter. One or more target cells reside in an area of a tissue or an organ (“target area”) of a subject. There may be one or more cells that reside adjacent to the target cells (“adjacent cells” or “neighboring cells”). Adjacent cells can be of the same or different types as the target cell. Adjacent cells can also comprise a transgene that is the same or different from the target cell transgene and is under the control of the same or different control element as the target transgene.

The target cell can be any cell that comprises a thermo generator, such as a chromophore.

By “thermo generator” herein is meant a molecule or complex that is capable of generating heat upon the application of electromagnetic radiation, such as laser. In some aspects, the thermo generator comprises a chromophore that can absorb certain spectrum of electromagnetic radiation, such as light.

By “chromophore” herein is meant a moiety of a molecule or complex that is responsible for the color of the molecule or complex. The thermo generator can be native to the cell, such as a pigment granule in a pigemented cell, or is introduced into the target cell using methods known in the art, such as direct administration of a pigment molecule, or using a gene known to express a pigment such as melanin, pheomelanin, eumelanin, neuromelanin, lipofuscin, or a carotenoid. For selective destruction of the target cells the chromophores or thermo generator should preferable be intracellular, such us melanosomes, an organelle containing melanin, a light-absorbing pigment found in the animal kingdom.

In some aspects, the chromophore comprises a pigment residing in a cell. A pigment is a material that changes the color of reflected or transmitted light as the result of wavelength-selective absorption. The RPE is composed of a single layer of hexagonal cells that are densely packed with pigment granules.

In general, the pigment granules in human RPE have a broad absorbance spectrum to protect human tissue. Thus, the pigment granules generate heat upon application of electromagnetic radiation, such as a laser.

Any cell that contains a thermo generator, either naturally occurring, or artificially introduced, is suitable as a target cell of the present invention. Preferably, the target cells reside in a part of the subject that is or can be made accessible to electromagnetic radiation. In some aspects, the target cell resides in a part of the subject is optically accessible such as a light (e.g. laser) can be applied to it. For example, a target cell can be located on the surface, such as the skin, or across a transparent space, such as the back of the eye. Alternatively the target cell could be located inside the body if light can be introduced to the location of the target cell, such as using optical fiber. It is known in the art that certain spectrum of the light, such infrared light, can penetrate certain depth of skin, thus can reach a target cell resides under the skin.

Suitable target cells include, but are not limited to, cells containing thermo generators, such as those containing chromophores or other pigments, such as RPE cells, melanocytes including normal melanocytes and melanoma, photoreceptors such as rods or cones, cells of the iris, liver, skin, blood, or other cells having thermo generators.

In some aspects, the target cell is a pigmented cell. Preferably, the pigmented cells reside in a tissue or location of a subject that is accessible to be radiated by electromagnetic radiation such as laser. In some aspects, the pigmented cells are in the eyes. Pigmented cells in the eye include retinal pigment epithelium, iris epithelium, trabecular meshwork epithelium, and cells in the pigmented choroid.

Various methods can be used to locate the target cells.

In some aspects, the target cells can be identified by co-localizing them with the transfected cells. This can be done, for example, by using a marker gene that is co-expressed and can be visualized or identified; e.g., with a fluorescent marker.

In some aspects, the marker gene encodes a fluorescent protein, such as green fluorescent protein (GFP), dsRED or any variants thereof that emits a color that is other than green.

In some aspects, the target cells can be identified by documentation of the area to which gene therapy was administered, e.g., with a drawing or photograph. In some aspects, this photograph is overlaid with a real-time image at the time of optical laser treatment.

### B. Transgenes

By “transgene” herein is meant a DNA sequence that is artificially introduced into a host cell or organism. Thus, the DNA may contain an expression cassette for siRNA, miRNA, or protein; this expression cassette may be artificially constructed, or may contain the same sequence as the endogenous gene of the host cell or organism. A transgene can be a DNA sequence that is not found in the genome of the host organism, or a DNA that is found in the genome of the host organism, but has been modified such that it has different sequence, under control of different regulatory element, or reside in a region of the genome that is not its original location. This non-native segment of DNA may retain the ability to produce RNA or protein in the transgenic organism, or it may alter the normal function of the transgenic organism's genetic code. A transgene can be either shRNA or other RNA expression constructs, the cDNA of a gene, or the genomic DNA of a gene.

A variety of genes can be used as transgene, including anti-angiogenic genes such as anti-VEGF aptamers, anti-VEGF antibodies or antibody fragments, VEGF soluble receptors such as sFlt-1, sFLT01, VEGF-Trap, an Fc fusion protein comprising a soluble FLT peptide or fragment including compositions that comprise a fusion protein, a soluble Tie-2 receptor, angiostatin, endostatin, pigment epithelium-derived factor (PEDF), TIMP-3, and other anti-angiogenic factors. It could also be neurotrophic or anti-apoptotic factors, such as glial derived neurotrophic factor (GDNF), ciliary neurotrophic factor (CNTF), Neurturin (NTN), neurotrophin-4 (NT-4), nerve growth factor (NGF), Bcl-2, Bcl-X1, platelet-derived growth factor receptors (PDGF-R) or human platelet-derived growth factor receptors (hPDGF-R) etc. Thus, the methods and compositions provided herein can be used to reverse the expression of these genes in a target cell. The present invention can also be used to regulate expression of factors important in genetic diseases of pigmented cells, such as RPE65, Mertk or other factors that could be intracellular to or secreted from target cells, such as rod-derived cone viability factor (RdCVF), Crumbs homolog 1 (CRB1), retinoschisin, retinitis pigmentosa GTPase regulator (RGPR)-interacting protein-1, peripherin, peripherin-2 (Prph2), a light-responsive opsin (ChR2; Chop2, L-opsin (OPN1LW), M-opsin (OPN1MW), S-Opsin (OPN1SW), rhodopsin (Rh1, OPN2, RHO), fibroblast growth factor 2, nurturin, epidermal growth factor, or a complement inhibitor.

### B1. Anti-VEGF

In some aspects, the transgene encodes an anti-VEGF protein, including, but not limited to the VEGF-binding fusion proteins disclosed in U.S. Pat. No. 7,635,474, and sFlt-1.

In some aspects, the anti-VEGF protein is sFlt-1 as provided herein.

A soluble truncated form of the VEGF receptor Flt-1, sFlt-1, is the only known endogenous specific inhibitor of VEGF. It is generated by alternative splicing and it lacks the membrane-proximal immunoglobulin-like domain, the transmembrane spanning region and the intracellular tyrosine-kinase domain. Binding of sFlt-1 to VEGF in vitro has been widely demonstrated [Barleon et al. 1997; Goldman et al. 1998; He et al. 1999]. The ability of sFlt-1 to inhibit VEGF-driven angiogenesis has attracted considerable attention for its potential clinical application [Aiello et al. 1995; Bainbridge et al. 2002; Goldman et al. 1998; Hasumi et al. 2002; Honda et al. 2000; Kendall and Thomas 1993; Kong et al. 1998; Lai et al. 2005; Lai et al. 2002; Liu et al. 2007; Mae et al. 2005; Mahasreshti et al. 2001; Shiose et al. 2000; Takayama et al. 2000; Zhang et al. 2005]. The angiostatic activity of sFlt-1 results from inhibition of VEGF by two mechanisms: i) sequestration of VEGF, to which it binds with high affinity, and ii) formation of inactive heterodimers with membrane-spanning isoforms of the VEGF receptors Flt-1 and KDR/Flk-1 [Kendall and Thomas 1993; Kendall et al. 1996].

sFlt-1 is a soluble truncated form of the Flt-1 and it is expressed endogenously. sFlt-1 is the only known endogenous specific inhibitor of VEGF. The angiostatic activity of sFlt-1 results from inhibition of VEGF by two mechanisms: i) sequestration of VEGF, to which it binds with high affinity, and ii) formation of inactive heterodimers with membrane-spanning isoforms of the VEGF receptors Flt-1 and Flk-1/KDR. In vitro binding assays showed that sFlt-1 binds VEGF with high affinity. sFlt-1 can also inhibit VEGF driven proliferation of human umbilical vein endothelial cells. In animal models for cancer, sFlt-1 inhibited tumor growth.

Preclinical studies have demonstrated that sFlt-1 binds to VEGF and appears to inhibit some of the ocular pathological changes associated with VEGF activity. In rat and nonhuman primate models, animals with transiently induced choroidal neovascularization (CNV), sFlt-1 significantly limits the neovascular changes and associated blood leakage which occurs. In a transgenic mouse model of retinal neovascularization with progressive neovascular changes and retinal degeneration, sFlt-1 can significantly limit the progression of neovascularization for long period of time.

By “sFlt-1 protein” herein is meant the naturally occurred sFlt-1 protein, as well as variant thereof, including, but not limited to functional fragments, insertion, deletion, substitution, etc. as long as the protein or polypeptide binds to the VEGF ligand and/or VEGF receptor, and/or functions as a direct VEGF blocker and/or dominant/negative inhibitor of VEGF activity.

### B2. Vectors

The transgene is generally introduced into the target cell using a vector, such as a recombinant virus or plasmid. The vectors can be replicative or non-replicative.

In some aspects, the vector that carries the transgene is non-replicative. In these aspects, both the target cell and cells that are adjacent the target cell comprises the transgene. After the target cell is destroyed upon radiation, the adjacent cells may divide to produce cells that occupy the location previously occupied by the target cell. However, as the vector is non-replicative, the daughter cells, or at least some of them, do not contain the transgene. In this way, the expression of transgene is not restored in the target area after the destruction of the target cell.

In some aspects, the vector that carries the transgene is replicative. These include vectors that stably integrate into the genome, such as retrovirus, lentivirus, phiC31 integrase, Sleeping Beauty, piggyBAC, and other transposases, as well as vectors that can replicate without genomic integration, such as the Epstein-Barr virus system. When this type of vector is used, expression of the transgene can be down-regulated if after the target cells are destroyed such as by laser, the target cells are replaced due to the spreading or migration, rather than division, of adjacent cells that are not destroyed.

### B3. Recombinant Viruses

In some aspects, the target gene, such as anti-VEGF proteins (e.g., sFlt-1 proteins) are expressed in the cells using gene therapy. The gene therapy uses a vector including a nucleotide encoding the target gene (such as sFlt-1). A vector (sometimes also referred to as gene delivery or gene transfer vehicle) refers to a macromolecule or complex of molecules comprising a polynucleotide to be delivered to the cell. The polynucleotide to be delivered may comprise a coding sequence of interest in gene therapy. Vectors include, for example, viral vectors such as adenoviruses, adeno-associated viruses (AAV), and retroviruses, liposomes and other lipid-containing complexes, and other macromolecular complexes capable of mediating delivery of a polynucleotide to a target cell. Some aspects of the present disclosure involve a vector comprising a recombinant nucleic acid.

The effective dose of the nucleic acid will be a function of the particular expressed protein, the particular condition to be targeted, the patient and his or her clinical condition, weight, age, sex, etc.

In some aspects, a delivery system is a recombinant viral vector that incorporates one or more of the polynucleotides therein, in some cases about one polynucleotide. Preferably, the viral vector used in the invention methods has a pfu (plague forming units) of from about 10⁸ to about 10¹⁴ pfu, or about 10⁸ to about 10¹⁴ viral particles or vector genomes. In aspects in which the polynucleotide is to be administered with a non-viral vector, use of between from about 0.1 nanograms to about 4000 micrograms will often be useful e.g., about 1 nanogram to about 100 micrograms.

Aspects of the invention also relate to expression vector constructs for the expression of the RNA oligonucleotides which contain hybrid promoter gene sequences and possess a strong constitutive promoter activity or a promoter activity which can be induced in the desired case.

Throughout this application, the term “expression construct” is meant to include any type of genetic construct containing a nucleic acid coding for gene products in which part or all of the nucleic acid encoding sequence is capable of being transcribed. The transcript may be translated into a protein, but it need not be. In certain aspects, expression includes both transcription of a gene and translation of mRNA into a gene product. In other aspects, expression only includes transcription of the nucleic acid encoding genes of interest.

In one aspect, the present invention provides a recombinant virus, such as adeno-associated virus (rAAV) as a vector to mediate the expression of sFlt-1.

The half-life of sFlt-1 protein is short. Expression of sFlt-1 protein by a viral vector such as rAAV vector, provides a long-term therapeutic effect.

In some aspects, a self-complementary vector (sc) is used. The self-complementary AAV vectors bypass the requirement for viral second-strand DNA synthesis and lead to greater rate of expression of the transgene protein, Wu, Hum Gene Ther. 2007, 18(2): 171-82.

In some aspects, several AAV vectors are generated to enable selection of the most optimal serotype, promoter, and transgene.

Vectors for use in the present invention include viral vectors, lipid based vectors and other non-viral vectors that are capable of delivering a nucleotide according to the present invention to the target cells. The vector can be a targeted vector, especially a targeted vector that preferentially binds to neoplastic cells, such as cancer cells or tumor cells. Viral vectors for use in the invention can include those that exhibit low toxicity to a target cell and induce production of therapeutically useful quantities of the transgene.

Suitable nucleic acid delivery systems include viral vector, typically sequence from at least one of an adenovirus-associated virus (AAV), helper-dependent adenovirus, retrovirus, herpes simplex virus, lentivirus, poxvirus, hemagglutinatin virus of Japan-liposome (HVJ) complex, Moloney murine leukemia virus, and HIV-based virus. Preferably, the viral vector comprises a strong promoter active in eukaryotic cells operably linked to the polynucleotide e.g., a cytomegalovirus (CMV) promoter.

Examples of viral vectors are those derived from adenovirus (Ad) or adeno-associated virus (AAV). Both human and non-human viral vectors can be used and the recombinant viral vector can be replication-defective in humans. The vector can comprise a polynucleotide having a promoter operably linked to a transgene and is replication-defective in humans.

To combine advantageous properties of two viral vector systems, hybrid viral vectors may be used to deliver a nucleic acid encoding a sFlt-1protein to a target tissue. Standard techniques for the construction of hybrid vectors are well-known to those skilled in the art. Such techniques can be found, for example, in Sambrook, et al, In Molecular Cloning: A laboratory manual. Cold Spring Harbor, N.Y. or any number of laboratory manuals that discuss recombinant DNA technology. Double-stranded AAV genomes in adenoviral capsids containing a combination of AAV and adenoviral ITRs may be used to transduce cells. In another variation, an AAV vector may be placed into a “gutless”, “helper-dependent” or “high-capacity” adenoviral vector. Adenovirus/AAV hybrid vectors are discussed in Lieber et al, J. Virol. 73:9314-9324, 1999. Retrovirus/adenovirus hybrid vectors are discussed in Zheng et al, Nature Biotechnol. 18:176-186, 2000.

Retroviral genomes contained within an adenovirus may integrate within the target cell genome and effect stable gene expression.

Replication-defective recombinant adenoviral vectors can be produced in accordance with known techniques. See, Quantin, et al., Proc. Natl. Acad. Sci. USA, 89:2581-2584 (1992); Stratford-Perricadet, et al., J. Clin. Invest, 90:626-630 (1992); and Rosenfeld, et al, Cell, 68:143-155 (1992).

Additionally some vectors include viral vectors, fusion proteins and chemical conjugates. Retroviral vectors include Moloney murine leukemia viruses and HIV-based viruses. One preferred HIV-based viral vector comprises at least two vectors wherein the gag and pol genes are from an HIV genome and the env gene is from another virus. DNA viral vectors are also preferred. These vectors include pox vectors such as orthopox or avipox vectors, herpesvirus vectors such as a herpes simplex I virus (HSV) vector [Geller, A. I. et al, J. Neurochem, 64: 487 (1995); Lim, F., et al, in DNA Cloning: Mammalian Systems, D. Glover, Ed. (Oxford Univ. Press, Oxford England) (1995); Geller, A. I. et al, ProcNatl. Acad. Set: U.S.A.:90 7603 (1993); Geller, A. I., et al, ProcNatl. Acad. Sci USA: 87:1149 (1990)], Adenovirus Vectors [LeGal LaSalle et al, Science, 259:988 (1993); Davidson, et al, Nat. Genet. 3: 219 (1993); Yang, et al, J. Virol. 69: 2004 (1995)] and adeno-associated Virus Vectors [Kaplitt, M. G., et al, Nat. Genet. 8:148 (1994)].

Other viral vectors that can be use in accordance with the present invention include herpes simplex virus (HSV)-based vectors. HSV vectors deleted of one or more immediate early genes (IE) are advantageous because they are generally non-cytotoxic, persist in a state similar to latency in the target cell, and afford efficient target cell transduction. Recombinant HSV vectors can incorporate approximately 30 kb of heterologous nucleic acid.

Retroviruses, such as C-type retroviruses and lentiviruses, might also be used in the invention. For example, retroviral vectors may be based on murine leukemia virus (MLV). See, e.g., Hu and Pathak, Pharmacol. Rev. 52:493511, 2000 and Fong et al, Crit. Rev. Ther. Drug Carrier Syst. 17:1-60, 2000. MLV-based vectors may contain up to 8 kb of heterologous (therapeutic) DNA in place of the viral genes. The heterologous DNA may include a tissue-specific promoter and a transgene. In methods of delivery to neoplastic cells, it may also encode a ligand to a tissue specific receptor.

Additional retroviral vectors that might be used are replication-defective lentivirus-based vectors, including human immunodeficiency (HlV)-based vectors. See, e.g., Vigna and Naldini, J. Gene Med. 5:308-316, 2000 and Miyoshi et al, J. Virol. 72:8150-8157, 1998. Lentiviral vectors are advantageous in that they are capable of infecting both actively dividing and non-dividing cells. They are also highly efficient at transducing human epithelial cells.

Lentiviral vectors for use in the invention may be derived from human and non-human (including SIV) lentiviruses. Examples of lentiviral vectors include nucleic acid sequences required for vector propagation as well as a tissue-specific promoter operably linked to a transgene. These former may include the viral LTRs, a primer binding site, a polypurine tract, att sites, and an encapsidation site.

A lentiviral vector may be packaged into any suitable lentiviral capsid. The substitution of one particle protein with another from a different virus is referred to as “pseudotyping”. The vector capsid may contain viral envelope proteins from other viruses, including murine leukemia virus (MLV) or vesicular stomatitis virus (VSV). The use of the VSV G-protein yields a high vector titer and results in greater stability of the vector virus particles.

Alphavirus-based vectors, such as those made from semliki forest virus (SFV) and sindbis virus (SIN), may also be used in the invention. Use of alphaviruses is described in Lundstrom, K., Intervirology 43:247-257, 2000 and Perri et al, Journal of Virology 74:9802-9807, 2000.

Recombinant, replication-defective alphavirus vectors are advantageous because they are capable of high-level heterologous (therapeutic) gene expression, and can infect a wide target cell range. Alphavirus replicons may be targeted to specific cell types by displaying on their virion surface a functional heterologous ligand or binding domain that would allow selective binding to target cells expressing a cognate binding partner. Alphavirus replicons may establish latency, and therefore long-term heterologous nucleic acid expression in a target cell. The replicons may also exhibit transient heterologous nucleic acid expression in the target cell.

Pox viral vectors introduce the gene into the cells cytoplasm. Avipox virus vectors result in only a short term expression of the nucleic acid. Adenovirus vectors, adeno-associated virus vectors and herpes simplex virus (HSV) vectors may be an indication for some invention aspects. The adenovirus vector results in a shorter term expression (e.g., less than about a month) than adeno-associated virus, in some aspects, may exhibit much longer expression. The particular vector chosen will depend upon the target cell and the condition being treated.

Adeno-associated viruses (AAV) are generally small non-enveloped single-stranded DNA viruses. They are non-pathogenic human parvoviruses and are dependent on helper viruses, including adenovirus, herpes simplex virus, vaccinia virus and CMV, for replication. Exposure to wild-type (wt) AAV is not associated or known to cause any human pathologies and is common in the general population, usually occurring in the first decade of life in association with an adenoviral infection [Blacklow et al. 1968; Moskalenko et al. 2000]. The wild-type AAV encodes rep and cap genes. The rep gene is required for viral replication and the cap gene is required for synthesis of capsid proteins. Through a combination of alternative translation start and splicing sites, the small genome is able to express four rep and three cap gene products. The rep gene products and sequences in the inverted terminal repeats (145 bp ITRs, which flank the genome) are critical in this process. To date, 11 serotypes of AAV have been isolated. AAV2 is the best characterized serotype and the serotype for which most gene transfer studies have been based upon.

Replication-defective recombinant adenoviral vectors can be produced in accordance with known techniques. See, Quantin, et al, Proc. Natl. Acad. Sci. USA, 89:2581-2584 (1992); Stratford-Perricadet, et al, J. Clin. Invest., 90:626-630 (1992); and Rosenfeld, et al, Cell, 68:143-155 (1992).

rAAVs are small non-enveloped single-stranded DNA viruses. They are non-pathogenic human parvoviruses and are dependent on helper viruses, including adenovirus, herpes simplex virus, vaccinia virus and CMV, for replication. Exposure to wild-type (wt) AAV is not associated or known to cause any human pathologies and is common in the general population, usually occurring in the first decade of life in association with an adenoviral infection.

Examples of viral vectors are those derived from adenovirus (Ad) or adeno-associated virus (AAV). Both human and non-human viral vectors can be used and the recombinant viral vector can be replication-defective in humans. Where the vector is an adenovirus, the vector can comprise a polynucleotide having a promoter operably linked to a gene encoding the light-sensitive transmembrane protein and is replication-defective in humans.

To combine advantageous properties of two viral vector systems, hybrid viral vectors may be used to deliver a nucleic acid encoding a sFlt-b 1protein to a target tissue. Standard techniques for the construction of hybrid vectors are well-known to those skilled in the art. Such techniques can be found, for example, in Sambrook, et al, In Molecular Cloning: A laboratory manual. Cold Spring Harbor, N.Y. or any number of laboratory manuals that discuss recombinant DNA technology. Double-stranded AAV genomes in adenoviral capsids containing a combination of AAV and adenoviral ITRs may be used to transduce cells. In another variation, an AAV vector may be placed into a “gutless”, “helper-dependent” or “high-capacity” adenoviral vector. Adenovirus/AAV hybrid vectors are discussed in Lieber et al, J. Virol. 73:9314-9324, 1999. Retrovirus/adenovirus hybrid vectors are discussed in Zheng et al, Nature Biotechnol. 18:176-186, 2000.

Retroviral genomes contained within an adenovirus may integrate within the target cell genome and effect stable gene expression.

In some aspects, the AAV is selected from the group consisting of: AAV1, AAV2, AAV2.5, AAV3, AAV4, AAV5, AAV6, AAV7, AAV8, AAV9, AAV10, AAV11, AAV 12, and hybrids or variants, including mutated or otherwise non-naturally occurring variants, thereof.

In some aspects, the present invention provides a recombinant virus comprising a vector and a human form of the truncated, soluble VEGF receptor 1 (sFlt-1) and is named rAAV.sFlt-1. The vector is a recombinant, replicative-deficient adeno-associated viral (rAAV) vector, of serotype 2.

AAV2 is the most characterized. rAAV2 has been shown to be able to mediate long-term transgene expression in the eyes of many species of animals. In rats, rAAV mediated reporter gene (green fluorescent protein) was still present at 18 months post injection. In monkeys, the same reported gene was present at 17 months post injection. Similarly, high sFlt-1 levels were present in the vitreous of rAAV.sFlt-1 injected monkey eyes at 12 months post injection.

rAAV.sFlt-1 has been tested in animal models for intraocular neovascular disorders. rAAV.sFlt inhibited or slowed the progression of neovascularization in animal models of corneal neovascularization and retinal neovascularization. A key finding was rAAV-mediated sFlt-1 inhibited neovascularization in a monkey model of choroidal neovascularization (model for the wet form of age related macular degeneration or AMD). In this study, the presence of the rAAV.sFlt-1 construct and the upregulation of sFlt-1 in the eyes did not affect the well-being or retinal function of the monkeys. There is no evidence to suggest any safety issues associated with systemic exposure to rAAV.sFlt-1. The overall positive findings and lack of toxicity of rAAV vectors in these studies, as well as the findings with rAAV.sFlt-1 in mammalian models of choroidal neovascularization/AMD provide extensive supporting data that the vector has a favorable safety profile when administered to the eye.

In addition, 3 clinical trials on Lebers Congenital Amaurosis (LCA) are being conducted in the UK and USA using the rAAV2 backbone. LCA is a rare inherited eye disease that appears at birth or in the first few months of life and it is characterized by nystagmus, sluggish or no pupillary responses, and severe vision loss or blindness. To date, no safety issues have been reported following injection of the rAAV2 construct into the subretinal space of 6 participants in these two trials. Both teams involved in the clinical trials concluded that their findings have supported further gene therapy studies in LCA patients.

Vectors can comprise components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities.

Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e., positive/negative) markers (see, e.g., Lupton, S., WO 92/08796, published May 29, 1992; and Lupton, S., WO 94/28143, published Dec. 8, 1994). Such marker genes can provide an added measure of control that can be advantageous in gene therapy contexts. A large variety of such vectors are known in the art and are generally available.

In many of the viral vectors compatible with methods of the invention, more than one promoter can be included in the vector to allow more than one heterologous gene to be expressed by the vector. Further, the vector can comprise a sequence which encodes a signal peptide or other moiety which facilitates expression of the light-sensitive transmembrane protein from the target cell.

The nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for polymerases. Much of the thinking about how promoters are organized derives from analyses of several viral promoters, including those for the HSV thymidine kinase (tk) and SV40 early transcription units. Promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 b.p. upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 b.p. apart before activity begins to decline. Depending on the promoter, it appears that individual elements can function either co-operatively or independently to activate transcription.

The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter.

In various aspects, the human cytomegalovirus (CMV) immediate early gene promoter, the SV40 early promoter, the Rous sarcoma virus long terminal repeat, β-actin, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized.

Selection of a promoter that is regulated in response to specific physiologic or synthetic signals can permit inducible expression of the gene product. For example in the case where expression of a transgene, or transgenes when a multicistronic vector is utilized, is toxic to the cells in which the vector is produced in, it may be desirable to prohibit or reduce expression of one or more of the transgenes. Examples of transgenes that may be toxic to the producer cell line are pro-apoptotic and cytokine genes.

The ecdysone system (Invitrogen, Carlsbad, Calif.) is one such system. This system is designed to allow regulated expression of a gene of interest in mammalian cells. It consists of a tightly regulated expression mechanism that allows virtually no basal level expression of the transgene, but over 200-fold inducibility. The system is based on the heterodimeric ecdysone receptor of Drosophila, and when ecdysone or an analog such as muristerone A binds to the receptor, the receptor activates a promoter to turn on expression of the downstream transgene high levels of mRNA transcripts are attained. In this system, both monomers of the heterodimeric receptor are constitutively expressed from one vector, whereas the ecdysone-responsive promoter which drives expression of the gene of interest is on another plasmid. Engineering of this type of system into the gene transfer vector of interest would therefore be useful. Cotransfection of plasmids containing the gene of interest and the receptor monomers in the producer cell line would then allow for the production of the gene transfer vector without expression of a potentially toxic transgene. At the appropriate time, expression of the transgene could be activated with ecdysone or muristeron A.

Another inducible system that would be useful is the Tet-Off™ or Tet-On™ system (Clontech, Palo Alto, Calif.). This system also allows high levels of gene expression to be regulated in response to tetracycline or tetracycline derivatives such as doxycycline. In the Tet-On™ system, gene expression is turned on in the presence of doxycycline, whereas in the Tet-Off™ system, gene expression is turned on in the absence of doxycycline. These systems are based on two regulatory elements derived from the tetracycline resistance operon of E. coli. The tetracycline operator sequence to which the tetracycline repressor binds, and the tetracycline repressor protein. The gene of interest is cloned into a plasmid behind a promoter that has tetracycline-responsive elements present in it. A second plasmid contains a regulatory element called the tetracycline-controlled trans activator, which is composed, in the Tet-Off™ system, of the VP 16 domain from the herpes simplex virus and the wild-type tertracycline repressor. Thus in the absence of doxycycline, transcription is constitutively on. In the Tet-On™ system, the tetracycline repressor is not wild type and in the presence of doxycycline activates transcription. For gene therapy vector production, the Tet-Off™ system would be preferable so that the producer cells could be grown in the presence of tetracycline or doxycycline and prevent expression of a potentially toxic transgene, but when the vector is introduced to the patient, the gene expression would be constitutively on.

In some circumstances, it may be desirable to regulate expression of a transgene in a gene therapy vector. For example, different viral promoters with varying strengths of activity may be utilized depending on the level of expression desired. In mammalian cells, the CMV immediate early promoter if often used to provide strong transcriptional activation. Modified versions of the CMV promoter that are less potent have also been used when reduced levels of expression of the transgene are desired. When expression of a transgene in hematopoietic cells is desired, retroviral promoters such as the LTRs from MLV or MMTV are often used. Other viral promoters that may be used depending on the desired effect include SV40, RSV LTR, HIV-1 and HTV-2 LTR, adenovirus promoters such as from the El A, E2A, or MLP region, AAV LTR, cauliflower mosaic Virus, HSV-TK, and avian sarcoma virus.

In a one aspect, tissue specific promoters are used to effect transcription in specific tissues or cells so as to reduce potential toxicity or undesirable effects to non-targeted tissues. For example, promoters such as the PSA, probasin, prostatic acid phosphatase or prostate-specific glandular kallikrein (hK2) may be used to target gene expression in the prostate.

In certain aspects of the invention, the use of internal ribosome entry site (IRES) elements is contemplated to create multigene, or polycistronic, messages. IRES elements are able to bypass the ribosome scanning model of 5′ methylated Cap dependent translation and begin translation at internal sites. IRES elements from two members of the picornavirus family (poliovirus and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

The selection of appropriate promoters can readily be accomplished. Preferably, one would use a high expression promoter. An example of a suitable promoter is the 763-base-pair cytomegalovirus (CMV) promoter. The Rous sarcoma virus (RSV) (Davis, et al, Hum Gene Ther 4:151 (1993)) and MMT promoters may also be used. Certain proteins can be expressed using their native promoter. Other elements that can enhance expression can also be included such as an enhancer or a system that results in high levels of expression such as a tat gene and tar element. This cassette can then be inserted into a vector, e.g., a plasmid vector such as, pUC19, pUC118, pBR322, or other known plasmid vectors, that includes, for example, an E. coli origin of replication. See, Sambrook, et al, Molecular Cloning: A Laboratory Manual, Cold Spring Harbor Laboratory press, (1989). Promoters are discussed infra. The plasmid vector may also include a selectable marker such as the p-lactamase gene for ampicillin resistance, provided that the marker polypeptide does not adversely effect the metabolism of the organism being treated. The cassette can also be bound to a nucleic acid binding moiety in a synthetic delivery system, such as the system disclosed in WO 95/22618.

In some aspects, the recombinant virus comprises a promoter selected from cytomegalovirus (CMV) promoter, Rous sarcoma virus (RSV) promoter, and MMT promoter.

In some aspects, the recombinant virus comprises a sequence encoding an antibiotic resistance gene, such as those provided herein.

In some aspects, the recombinant virus comprises a sequence encoding a replication origin sequence, such as those provided herein.

In some aspects, the recombinant virus comprises an enhancer, such as those provided herein.

In some aspects, the recombinant virus comprises a chimeric intron, such as those provided herein and disclosed in U.S. Pat. No. 7,635,474.

In some aspects, the recombinant virus comprises a poly A sequence, such as those provided herein (e.g. SV40 poly A sequence.).

In some aspects, the recombinant virus comprises a human sFlt-1 protein or a functional fragment thereof.

In some aspects, the recombinant virus comprises a regulatory nucleic acid fragment that is capable of directing selective expression of the sFlt-1 protein in an eye cell.

### C. Down-Regulation of Gene Expression Using Electromagnetic Radiation

In some aspects, electromagnetic radiation is applied to the target cell for a suitable period of time to generate heat to destroy the target cell, thereby modulating expression of the transgene in the subject.

In this method, target cells are damaged, killed, destroyed, or vaporized by the application of electromagnetic radiation, such as laser, to eliminate transgene expression from those target cells. Thus, the transgene is down-regulated in cells such as pigmented cells by selective damage or destruction of a fraction of the transfected cells. This is achieved by controlling the energy absorbed by target cells, such as through a proper combination of the duration and power or intensity of electromagnetic radiation applied. The power or intensity of the beam is adjusted with beam size and/or wavelength shift. In some aspects, the method comprises using laser exposures short enough to prevent significant thermal destruction of nearby or adjacent cells.

In some aspects, using the various methods provided herein (e.g. using electromagnetic radiation, radio frequency ablation, or ultrasound) transgene expression is reduced by 10, 15, 20, 25, 30, 35, 40, 45, 55, 60% or more in comparison to the expression level without the treatment. In additional aspects, the percent of transfected tissue that may be targeted with a single application of the method provided herein may range from 0% to 90%. This range of coverage of the transfected tissue makes it possible to achieve a specific target level of transgene down regulation, from about 0% to about 100%, by combining a range of coverage with multiple applications of the method provided herein. For example, a reduction in transgene expression of about 80% could be achieved with four applications of 20% transfected tissue coverage, two applications of 40% transfected tissue coverage or one application of 50% transfected tissue coverage followed by a second application of 30% transfected tissue coverage.

In some aspects, the removed cells are not replaced, and transgene expression remains at the lower level. In other aspects the removed cells are replaced by cell division in the surrounding tissue, but the gene therapy vector does not replicate, thereby reducing the overall level of transgene expression. In some aspects, laser settings are used that are capable of targeting transduced cells, but are not destructive to surrounding tissue. In one such example, settings are used that do not target adjacent cells; for example line scanning is used to target cells in the area of line scanning, but adjacent cells are not targeted. In another example, electromagnetic radiation settings are used that are absorbed by target cells at a greater level compared to adjacent cells, for example settings where electromagnetic radiation is preferentially absorbed by RPE cells compared to the neurosensory retina.

By “electromagnetic radiation” or “EMR” herein is meant a form of energy emitted and absorbed by charged particles, which exhibits wave-like behavior as it travels through space. EMR is classified according to the frequency of its wave. The electromagnetic spectrum, in order of increasing frequency and decreasing wavelength, consists of radio waves, microwaves, infrared radiation, visible light, ultraviolet radiation, X-rays and gamma rays. Preferably, the electromagnetic radiation of the present invention has a wave length from 300 to 3000 nm, more preferably 400 to 1000 nm.

To down-regulate expression of the transgene in target cells (such as transfected RPE cells) these cells can be selectively destroyed by a short-pulse EMR such as laser. The removed target cells are either not replaced, reducing the level of transgene expression, or replaced by cell division of the surrounding undamaged cells. When a non-replicative vector is used to introduce the transgene, the gene therapy vector does not replicate, thereby reducing the overall level of transgene expression in the subject.

In some aspects, selective destruction of target cells is achieved using microsecond laser exposures. Without being bound by any particular theory, it is believed that microsecond pulses produce intracellular microbubbles around light-absorbing melanosomes that leads to selective death of RPE cells, while the surrounding retinal temperature remains sublethal. Selective RPE destruction without photoreceptor damage may be achieved with an Nd:YLF laser using pulse durations of 1.7 μs. OCT imaging in human patients has demonstrated unaffected neural retina and RPE thinning at 1 hour and normal neural retina and RPE at 1 year. At energies corresponding to selective damage of the RPE, no immediate ophthalmoscopically visible retinal lesions are produced.

In some aspects, microsecond exposures are produced with a continuous scanning laser. Exposure time in this approach is determined by the laser spot size on the target tissue divided by the scanning speed of the laser. With appropriately high scanning speed, microsecond-range exposures are produced, resulting in selective target cell damage.

For example, with an aerial beam diameter of 100 μm and a velocity of 6.6 m/s, the exposure duration is 15 μs. During such short time heat diffusion remains confined to the cellular scale, thereby allowing rapid heating and selective destruction of the target pigmented cells, while avoiding damage to the surrounding non-pigmented tissue.

With sufficiently short pulses the heat produced by light absorption in the pigmented cells does not have time to escape from the absorption sites during the laser pulse, which allows for selective heating of the target cells while avoiding the thermal damage to the surrounding tissue. This condition is called heat confinement. For example, during 1 μs pulses the 1 μm sized melanosomes can be selectively heated to temperatures exceeding vaporization threshold. Vapor microbubbles destroy the pigmented cells, while no damage is produced to the surrounding cells. With the target size of L, the thermal confinement time ρ=L²/4α, where α is thermal diffusivity of tissue (temperature conductivity α≈0.14 mm²/s). For example, in water with L=1 μm, pulse duration generally should not exceed 1.7 μs. For a target size of L=6 μm, t=61 μs.

An example of selective destruction of RPE cells produced by scanning laser in a rabbit eye without damage to photoreceptors is provided in the Example.

An example for control expression of the trans-gene in the laser-treated RPE using a gene encoding Green Fluorescent Protein (GFP) is also provided in the Example.

By adjusting the treatment pattern density one can precisely regulate the extent of reduction of the gene expression. Since the treatment does not harm overlying photoreceptors this procedure does not generally adversely affect functional vision. As RPE restores its continuity within a few days, additional scanning can be applied later to further reduce gene expression, if necessary, and can be applied without adverse effects to the retina. Multiple applications of selective cell damage for further reduction of the number of transfected cells. In some aspects, a second scanning is applied 3, 4, 5, 6, 7, 8, 9, 10 days or anytime greater than 10 days, such as 30, 60, or 90 days, after the first scanning. Additional one, two, three, four or a greater number of additional procedures can also be applied after the second scanning. In some aspects, the subsequent scanning is applied 3, 4, 5, 6, 7, 8, 9, 10 days or anytime greater than 10 days, such as 30, 60, or 90 days, after the previous scanning. In some aspects, subsequent scanning is carried on area where the previous scanning was not targeted in order to maximize the reduction.

In some aspects, the electromagnetic radiation of the present invention is produced by laser.

The wavelength of the laser is selected based on the absorbance spectrum of the thermo generator such as a desirable amount of heat can be generated. In some aspects, the laser has a wavelength from 300 to 3000 nm, more preferably 400 to 1000 nm.

In some aspects, the laser beam has a diameter or width between 10 μm and 1000 μm, preferable between 50 μm and 200 μm. In some aspects, the laser beam has a diameter or width of about 10 to 20, 30, 40, 50, 60, 70, 80, 90, 100, 150, 200, 250, 300, 400, 500, 600, 700, 800, 900, or 1,000 μm, preferably about 50, 100, 150, or 200 μm.

In some aspects, the laser has a power of about 500 mW to 5,000 mW, preferably about 600, 700, 800, 900, 1,000, 1,100, 1,200, 1,400, 1,500, 1,600, 1,800, 2,000, 2,500, 3,000, or 4,000 mW.

Without being bound by any particular theory, cell damage or destruction is a function of the total energy absorbed by the cell, which depends on properties of the cell, such as cell type, cell size, cell absorbance rate, color and density of the chromophore that absorbs the energy, as well as the tolerance level of the cell to heat damage. As such, various combinations of exposure times (such as controlled by scan speed or pulse duration) and beam size can be used to selectively damage or destroy the RPEs. In some aspects, the pulsed electromagnetic radiation is applied at radiant exposures ranging from 5, to 10, 20, 30, 50, 80, 100, 200, 300 and to 500 J/cm².

In some aspects, the electromagnetic radiation, such as laser, is applied in pulse. In general, the pulse has a frequency ranging from 1 to 1000 Hz.

In some aspects, the duration of applying the pulsed electromagnetic radiation is less than 100 μs.

In some aspects, the electromagnetic radiation is applied by scanning. In these aspects, the laser is scanned in a variety of pattern to obtain partial coverage of the target area. The scanning can be carried as parallel lines with equal or different distances between the lines, or in co-centric circles.

The size or percentage of the target area covered by the scanning laser varies. In some aspects, the laser is scanned to regulate transgene in the eye and the target area is the retina. In general, about 10 to 20, 30, 40, 50, 60, 70, 80, or 90 percentage of the retina is covered, with a spot size of from 10 to, 20, 30, 40, 50, 60, 80, 100, 200, 300, 400, 500, 600, 700, 800, 900, or 1000 μm.

In some aspects, the scanning velocity of said beam is between 0.5 m/s and 50 m/s, preferably between 1 m/s and 2, 3, 4, 5, 6, 7, 8, 9, 10, 15, 20, 25, 30, 35, or 40 m/s.

In some aspects, the scanning beam has a dwell time less than 100 μs, preferably below 20 μs.

In some aspects, when a pulsed laser is used, the number of pulses applied is from 1 to 1000.

In some aspects, target cells are selectively targeted with nanoparticles comprising one or more targeting moieties which form nanoparticle clusters thereon or therewithin. Pulsed electromagnetic radiation, e.g., optical radiation, having a wavelength spectrum selected for a peak wavelength near to or matching a peak absorption wavelength of the nanoparticles selectively heats the Nano particulates thereby generating vapor microbubbles around the clusters causing damage to the target cells without affecting any surrounding medium or normal cells or tissues. See U.S. Pat. No. 7,999,16, which is incorporated by reference in its entirety.

### D. Down-Regulation of Gene Expression by Radio Frequency Ablation

In some aspects, radio frequency ablation (RFA) is used to damage or destroy the target cells.

During RFA part of the electrical conduction system of the heart, tumor or other target tissue is ablated using the heat generated from the high frequency alternating current to treat a medical disorder. RF generally does not directly stimulate nerves or heart muscle and can therefore often be used without the need for general anesthetic. RFA procedures are generally performed under image guidance (such as X-ray screening, CT scan or ultrasound) by an interventional pain specialist (such as an anesthesiologist), interventional radiologist or a cardiac electrophysiologist, a subspecialty of cardiologists. In some aspects, RF ablation is performed with a catheter or wire coil and which allows access to the heart, the GI tract, the spine, the brain, the sinuses, the lungs and much of the arterial system. See U.S. Pat. No. 5,383,917, US 2011/0270256 A1 and US 2009/0112201 A1, each is incorporated by reference in its entirety.

### E. Down-Regulation of Gene Expression by Ultrasound

In some aspects, ultrasound is used to damage or destroy the target cells. In these aspects, a high power acoustic wave generating apparatus is used to generate acoustic wave that damage or destroy target cells. Preferably, the ultrasound is applied as a high intensity focused ultrasound energy beam to the target area and target cells. See U.S. Pat. Nos.: 5,219,401 and 8,137,274, each is incorporated by reference in its entirety.

III. Modulation of Gene Expression through Heat Shock Protein Pathway

In another aspect, the present invention provides a method for modulating gene expression in a target cell by electromagnetic radiation. A heat-responsive promoter is used, which enables direct or indirect up regulation of transgene expression.

This method uses a non-destructive laser irradiation regime, raising the temperature of cells to stimulate increased transgene expression in an area transduced by gene therapy. A promoter is used that is responsive to heat in the range produced by sub-lethal radiation, such as laser. In this aspect of the invention, laser exposure produces heat stress below the threshold of cell destruction, thereby stimulating expression of the transgene while avoiding irreversible tissue damage. The method comprises: (a) expressing a transgene in a target cell, wherein the target cell comprises: (i) a polynucleotide encoding heat-responsive promoter that controls expression of the transgene; and (iii) a thermogenerator that is capable of generating heat upon application of an electromagnetic radiation; and (b) applying the electromagnetic radiation to the target cell for a suitable period of time to increase the expression of the transgene.

In this method, the target cell, thermogenerator, transgene, and vector are similar to those used in the method of controlling gene expression by destroying the target cell as provided herein, except that: (1) the transgene is controlled by a heat-responsive promoter; and (2) the electromagnetic radiation is sub-lethal to the target cells.

Preferably, target cell comprises a thermo generator as provided herein.

In some aspect, the target cell does not contain a thermo generator, but resides in the vicinity of a thermo generator that may or may not resides in an adjacent cell. For example, gas bubbles could be introduced into the body adjacent to target cells or tissues that could function as thermogenerators.

### A. Heat-Responsive Promoter

In some aspects, the transgene is controlled by a heat-responsive promoter.

By “heat-responsive promoter” herein is meant a promoter that is responsive to a change of temperature so that the expression of a transgene controlled by the promoter is changed accordingly. Such promoters include members of the heat shock protein family (e.g., HSP70) as well as other promoters known to respond to heat-induced stress (e.g., CMV promoter, see Pshenichkin, S. et al).

Any heat-inducible promoter can be used in accordance with the methods of the present invention, including but not limited to a heat-responsive element in a heat shock gene (e.g., hsp20-30, hsp27, hsp40, hsp60, hsp70, andhsp90). See Easton et al. (2000) Cell Stress Chaperones 5(4):276-290; Csermely et al. (1998) Pharmacol Ther 79(2): 129-1 68; Ohtsuka & Hata (2000) Int J Hyperthermia 16(3):231-245; and references cited therein. Sequence similarity to heat shock proteins and heat-responsive promoter elements have also been recognized in genes initially characterized with respect to other functions, and the DNA sequences that confer heat inducibility are suitable for use in the disclosed gene therapy vectors. For example, expression of glucose-responsive genes (e.g., grp94, grp78, mortalin/grp75) (Merrick et al. (1997) Cancer Lett 119(2): 185-190; Kiangetal. (1998)FASEB J 12(14): 1571-16-579), calreticulin (Szewczenko-Pawlikowski et al. (1997) Mol Cell Biocheml77(l -2): 145-1 52); clusterin (Viard et al. (1999) J Invest Dermatol 112(3):290-296; Michel et al. (1997) Biochem J 328(Ptl):45-50; Clark & Griswold (1997) J Androl 18(3):257-263), histocompatibility class I gene (HLA-G) (Ibrahim et al. (2000) Cell Stress Chaperones 5(3):207-218), and the Kunitz protease isoform of amyloid precursor protein (Shepherd et al. (2000) Neuroscience 99(2):31 7-325) are upregulated in response to heat.

In the case of clusterin, a 14 base pair element that is sufficient for heat-inducibility has been delineated (Michel et al. (1997) Biochem J 328(Ptl):45-50). Similarly, a two sequence unit comprising a 10- and a 14-base pair element in the calreticulin promoter region has been shown to confer heat-inducibility (Szewczenko-Pawlikowski et al. (1997) Mol Cell Biochem 177(1 -2): 145-1 52). A suitable promoter can incorporate factors such as tissue-specific activation. For example, hsp70 is transcriptionally impaired in stressed neuroblastoma cells (Drujan & De Maio (1999) 12(6):443-448). The mortalin promoter, which is upregulated in human brain tumors (Takano et al. (1997) Exp Cell Res 237(1):38-45).

### B. Heat-shock Protein Inducible Promoter

In some aspects, rather than being controlled by a heat-inducible promoter directly, the transgene is controlled by a promoter that is inducible by a heat-shock protein. In these aspects, sub-lethal heating of the transfected cells to cause expression of the endogenous heat-shock proteins which in turn promote the transgene expression, either directly or indirectly.

### C. Application of Laser

To obtain optimal control of transgene expression, the power of electromagnetic radiation is first titrated to obtain the cellular damage threshold and then is lower. For example, when laser is used, the laser is titrated first to produce a visible retinal lesion, and then its power or pulse duration is reduced to be below the cellular damage threshold.

In some aspects, the electromagnetic radiation of the present invention is produced by laser.

The laser can be generated by any laser sources that are available as long as the source can generate the laser beam as provided herein and provide either pulsed laser or laser scanning at the provided herein. Exemplary laser sources are: Nd:YAG or other solid state laser, Argon or other gas laser, diode laser.

In some aspects, an initial power of 10 mW to 3000 mW is used for titration and the laser is delivered at a velocity of from 1 to 10 m/s, to cover an area from 0.01 to 1 mm² or line from 0.5 to 5 mm in length.

In some aspects, the laser has a has a wave length from 300 to 3000 nm, more preferably 400 to 1000 nm.

In some aspects, the laser has a beam width of about 10 to 20, 30, 40, 50, 60, 70, 80, 90, 100, 150, 200, 250, 300, 400, 500, 600, 700, 800, 900, or 1,000 μm, preferably about 30 to 200 μm.

In some aspects, the laser has a power of about 5 mW to 500 mW, preferably about 5, 10, 15, 20, 25, 30, 35, 40 mW or more. The maximum power of the laser preferably is lower than the cellular damage threshold as determined by titrating the power of the laser.

In some aspects, the electromagnetic radiation, such as laser, is applied in pulses. In general, the pulse has a frequency from 1 Hz to 10 kHz.

In some aspects, the duration of applying the electromagnetic radiation is less than 100 s

In some aspects, the electromagnetic radiation is applied by scanning. In general, the laser is scanned only once, but in some aspects, the laser scanning is carried 1 , 2, 3, 4, or more times.

In some aspects, the scanning velocity of said beam is from 0.1 m/s to 100 m/s

### IV. Devices

In another aspect, the present invention provides an apparatus for regulating transgene expression in target cells. The apparatus comprises: (a) a laser device that is capable of delivering a laser beam under predetermined settings; and (b) a module that is configured for or is capable of, identifying cells that have been targeted by gene therapy, such as a drawing showing the location of target cells.

The laser device comprises a light source that can generate laser of suitable beam size, power strength, and frequency if pulsed laser is used.

The apparatus also includes a way to identify target cells, such as a delivery module that deliver the laser beam to the target area and is capable to focus the laser at a location with suitable accuracy. In some aspects, this module would allow a photograph to be overlaid with a real-time image at the time of optical laser treatment.

In some aspects, the apparatus is configured to deliver the laser in a line-scanning matter with high speed, such as between 0.5 m/s and 50 m/s, preferably between 1 m/s and 2, 3, 4, 5, 6, 7, 8, 9, 10, 15, 20, 25, 30, 35, or 40 m/s.

The apparatus further comprises a control module that is able to control the laser source and the delivery module to deliver proper laser beam to the target area.

The control module generally comprise a non-transient computer readable medium that contains a software to control the operation of the apparatus. The software contains code that when executed can control the apparatus.

The apparatus may also comprise input and out device, such as keyboard and monitor, and storage medium such as hard drive or solid state drive.

### V. Gene Therapy

The compositions and methods of the present invention are particularly useful in gene therapy in various tissues or organs, such as the eye and the retina of the eye.

Recent advances, including demonstration of safety and efficacy in human trials, have ushered in a new era of promise for gene therapy of retinal diseases. The retina provides a favorable environment for gene therapy applications as an isolated organ, with the minimal risk of vector or product spread to unwanted tissues, and the eye enjoys immune privilege. Specifically, adeno-associated virus (AAV) has emerged as a proven vector for clinical applications of ocular gene therapy, and represents a promising strategy for long-term, sustained secretion of proteins in the eye. Clinical experience with rAAV vector for an inherited retinal degenerative condition (Leber congenital amaurosis, LCA) demonstrated that rAAV2 is safe, with no dose-limiting toxicity observed. Patients showed visual function improvement, sustained now beyond 4 years. Several additional ocular gene therapy programs are advancing toward the clinic, including the treatment of age-related macular degeneration (AMD), retinitis pigmentosa, retinoschisis, Stargardt's disease, and further trials in LCA.

AMD is the leading cause of visual loss in patients >50 years old, with 7.5 million people likely to be affected by AMD-related visual impairment in the US alone by 2020. Current standard of care for treatment of wet AMD and DME is monthly intravitreal injections of ranibizumab, which inhibits VEGF-A, thereby slowing progression neovascularization and reducing macular edema. Frequent injections are burdensome for patients and physicians, are associated with very high cost to the healthcare system, and carry cumulative risk of ocular infection with potentially devastating consequences. In 2009, 1,270,836 intravitreal injections for eye diseases cost Medicare and Medicaid more than $2B, which is expected to grow rapidly as more indications are approved and the population ages. Such an immense financial burden on healthcare and patients signifies an urgent need to develop more long-lasting and cost-effective treatments for retinal diseases.

Gene therapy approaches are being developed to treat retinal neovascular diseases by using viral transfection of ocular cells to create a “biofactory” that secretes a soluble therapeutic protein. The leading program in development uses rAAV encoding for sFlt-1, which is a highly potent and naturally occurring anti-angiogenic peptide that binds and inactivates VEGF. Subretinal delivery of rAAV.sFlt-1 prevents and reverses progression of ocular neovascularization in mice and non-human primates, and is currently the subject of a Phase ½ clinical trial for wet AMD (NCTO1494805). However, one of the challenges for the gene therapy in general, and for the “biofactory” approach in particular, is a potential need to down-regulate gene expression in the case of total resolution of retinal condition, or if patient has adverse reaction to the secreted protein, such as a stroke or other thromboembolic adverse event. In such cases it would be desirable to “turn off” or down-regulate transgene expression.

Significant efforts have been dedicated to developing genetic regulatory systems, such as the tetracycline or rapamycin-based inducible systems, secondary transfection with an inhibitor gene and more recently, a light switchable transgene system was developed. However, so far none of these approaches works satisfactorily for clinical applications to retinal therapy. The method provided herein is used for optical down-regulation of the transgene expression in retinal pigment epithelium (RPE) using a rapid line scanning laser. Microsecond exposures produced by a rapid line scanning laser vaporize malanosomes and thereby selectively destroy RPE cells without damage to overlying photoreceptors and underlying Bruch's membrane. RPE continuity is restored within days due to stretching, migration and proliferation of adjacent RPE, but since the transgene is not integrated into the nucleus, it does not replicate, reducing the amount of transgene in the RPE. The extent of reduction in transgene expression is precisely controlled by adjusting the density of the laser scanning pattern, and by repeated treatment. This technique provides a robust and convenient way to control gene expression in clinical applications of gene therapy targeting cells such as RPE.

Microsecond exposures can also be produced with a continuous scanning laser. Exposure time in this approach is determined by the laser spot size on the target tissue divided by the scanning speed of the laser. With appropriately high scanning speed, microsecond-range exposures can be produced, resulting in selective RPE damage. For example, with an aerial beam diameter of 100 μm and a velocity of 6.6 m/s, the exposure duration is 15 μs. During such short time heat diffusion remains confined to the cellular scale, thereby allowing rapid heating and selective destruction of the target pigmented cells, while avoiding damage to the surrounding photoreceptors and Bruch's membrane.

Since after the rAAV2-mediated transfection the transgene is not integrated into the cell genome the proliferating RPE cells will not duplicate this gene. Therefore selective destruction of RPE cells can reduce the amount of secreted protein even though RPE cells proliferate and restore continuity of the RPE layer. Destroying a fraction of transfected RPE cells should reduce the amount of secreted protein accordingly. Dosimetry of the down-regulation can be therefore controlled by the density of the line scanning pattern.

One major area of gene therapy research in recent years is in the retina, which provides a favorable environment for gene therapy applications. As an isolated organ, the risk of vector or product spread to unwanted tissues, such as the germ line, are minimal. The eye is known to be partially immune privileged. In addition, its small size allows local delivery to a small area to achieve a therapeutic effect, and the small amount of vector needed makes scale-up and production tractable and commercially feasible. In particular, adeno-associated virus (AAV) has emerged as a proven vector for clinical applications of ocular gene therapy, and represents a promising strategy for long-term, sustained delivery of proteins to the eye. AAV has several desirable features as a gene therapy vector. AAV is non-pathogenic and is not associated with disease in humans. AAV has low immunogenicity. In addition, rAAV is non-integrating and capable of long-term expression in non-dividing cells. Recent clinical experience with AAV for an inherited retinal degenerative condition has been positive: In three independent Phase I clinical trials, rAAV2 expressing RPE65 was delivered via subretinal injection in patients with the rare disorder Leber's Congenital Amaurosis 2, a form of early childhood blindness caused by deficiency of the isomerase RPE65. Initial results from all three studies indicate that rAAV2 is safe, with no dose-limiting toxicities observed. Across the three trials, no serious adverse events were observed. The vector was well-tolerated, with no humoral immune reactions to AAV2 and no T cell-mediated response, although one patient had a borderline increase in AAV2-stimulated lymphocyte proliferation. Patients in all three studies showed visual function improvement, as measured by pupillometry, light sensitivity, microperimetry, visual mobility, and visual acuity. Improvements were sustained and long term, with observations now beyond 4 years, including safe and effective re-administration to the contralateral eye. If the animal models are indicative, therapeutic benefit may be sustained over the very long term: in a naturally-occurring canine model that mimics the human disease, treatment with rAAV2.RPE65 has shown long-term rescue of functional vision beyond 7.5 years, with ongoing observation. These recent successes in the clinic have generated excitement and momentum for AAV-mediated gene therapy in ocular applications. Indeed, several ocular gene therapy programs have advanced toward the clinic, including in age-related macular degeneration, retinitis pigmentosa, retinoschisis, Stargardt's disease, and further trials in LCA.

This method utilizes novel approach to retinal laser therapy allowing selective destruction of RPE cells without damage to overlying photoreceptors or underlying Bruch's membrane. The ablated RPE cells are replaced by either stretching and migration of the adjacent cells or by cell division of the surrounding RPE. Since rAAV2 vector does not replicate, the final result of these processes is the reduction of the overall level of transgene expression in the treated RPE areas. By adjusting the treatment pattern density one can precisely regulate the extent of reduction of the gene expression. Furthermore, since RPE restores its continuity within a few days, additional scanning can further reduce gene expression without incremental adverse effects to the retina. If our approach proves successful clinically, it could be directly applied to programs that focus on transfection of RPE cells with anti-angiogenic agents for treatment of proliferative AMD. This technology can also be applied to any therapy using non-integrated vectors transfecting RPE cells, and will significantly enhance the potential of gene therapy programs in clinical development now and in the future.

The present invention can be applied to a number of applications where pigmented cells are targeted for gene therapy, including treatment for wet age-related macular degeneration, dry age-related macular degeneration, diabetic retinopathy (proliferative and non-proliferative), macular edema (diabetic or other origin), choroideremia, Leber's Congenital Amaurosis, retinoschisis, retinitis pigmentosa, macular telangectasia, Usher's syndrome, Stargardt's disease, and other diseases of the outer retina.

### REFERENCES

1. Sheridan C. Gene therapy finds its niche. Nat Biotechnol 2011;29(2):121-8.
2. Bennett J. Gene therapy for retinitis pigmentosa. Curr Opin Mol Ther 2000;2(4):420-5.
3. Dejneka N S, Bennett J. Gene therapy and retinitis pigmentosa: advances and future challenges. Bioessays 2001;23(7):662-8.
4. Dejneka N S, Rex T S, Bennett J. Gene therapy and animal models for retinal disease. Dev Ophthalmol 2003;37:188-98.
5. Provost N, Le Meur G, Weber M, et al. Biodistribution of rAAV vectors following intraocular administration: evidence for the presence and persistence of vector DNA in the optic nerve and in the brain. Mol Ther 2005;11(2):275-83.
6. Streilein J W. Unraveling immune privilege. Science 1995;270(5239): 1158-9.
7. Wenkel H, Streilein J W. Analysis of immune deviation elicited by antigens injected into the subretinal space. Invest Ophthalmol Vis Sci 1998;39(10): 1823-34.
8. Wenkel H, Streilein J W. Evidence that retinal pigment epithelium functions as an immune-privileged tissue. Invest Ophthalmol Vis Sci 2000;41(11):3467-73.
9. Maguire A, Simonelli F, Pierce E, et al. Safety and efficacy of gene transfer for Leber's congenital amaurosis. N Engl J Med 2008;358(21):2240-8.
10. Hauswirth W, Aleman T, Kaushal S, et al. Treatment of leber congenital amaurosis due to RPE65 mutations by ocular subretinal injection of adeno-associated virus gene vector: short-term results of a phase I trial. Hum Gene Ther 2008;19(10):979-90.
11. Bainbridge J, Smith A, Barker S, et al. Effect of gene therapy on visual function in Leber's congenital amaurosis. N Engl J Med 2008;358(21):2231-9.
12. Maguire A M, Simonelli F, Pierce E A, et al. Safety and efficacy of gene transfer for Leber's congenital amaurosis. N Engl J Med 2008;358(21):2240-8.
13. Bainbridge J W, Smith A J, Barker S S, et al. Effect of gene therapy on visual function in Leber's congenital amaurosis. N Engl J Med 2008;358(21):2231-9.
14. Bennett J, Ashtari M, Wellman J, et al. AAV2 gene therapy readministration in three adults with congenital blindness. Sci Transl Med 2012;4(120):120ra15.
15. Lai C M, Estcourt M J, Himbeck R P, et al. Preclinical safety evaluation of subretinal AAV2.sFlt-1 in non-human primates. Gene Ther 2011.
16. Maclachlan T K, Lukason M, Collins M, et al. Preclinical safety evaluation of AAV2-sFLT01-a gene therapy for age-related macular degeneration. Mol Ther 2010;19(2):326-34.
17. Liu M M, Tuo J, Chan C C. Gene therapy for ocular diseases. Br J Ophthalmol 2010.
18. Mitchell P, Annemans L, White R, et al. Cost effectiveness of treatments for wet age-related macular degeneration. PharmacoEconomics 2011;29(2): 107-31.
19. Stieger K, Belbellaa B, Le Guiner C, et al. In vivo gene regulation using tetracycline-regulatable systems. Advanced drug delivery reviews 2009;61(7-8):527-41.
20. Goverdhana S, Puntel M, Xiong W, et al. Regulatable gene expression systems for gene therapy applications: progress and future challenges. Molecular therapy: the journal of the American Society of Gene Therapy 2005; 12(2): 189-211.
21. Pollock R, Issner R, Zoller K, et al. Delivery of a stringent dimerizer-regulated gene expression system in a single retroviral vector. Proceedings of the National Academy of Sciences of the United States of America 2000;97(24): 13221-6.
22. Wang X, Chen X, Yang Y. Spatiotemporal control of gene expression by a light-switchable transgene system. Nature Methods 2012;9(3):266-9.
23. Framme C, Alt C, Schnell S, et al. Selective targeting of the retinal pigment epithelium in rabbit eyes with a scanning laser beam. Investigative ophthalmology &amp; visual science 2007;48(4): 1782-92.
24. Paulus Y M, Jain A, Nomoto H, et al. Selective retinal therapy with microsecond exposures using a continuous line scanning laser. Retina 2011;31(2):380-8.
25. Khani S C, Pawlyk B S, Bulgakov O V, et al. AAV-mediated expression targeting of rod and cone photoreceptors with a human rhodopsin kinase promoter. Investigative ophthalmology &amp; visual science 2007;48(9):3954-61.
26. Schuele, G., Rumohr, M., Huettmann, G. & Brinkmann, R. RPE damage thresholds and mechanisms for laser exposure in the microsecond-to-millisecond time regimen. Invest Ophthalmol Vis Sci 46, 714-719 (2005).
27. Brinkmann, R. et al. Origin of retinal pigment epithelium cell damage by pulsed laser irradiance in the nanosecond to microsecond time regimen. Laser Surg Med 27, 451-464 (2000).
28. Brinkmann, R., Roider, J. & Birngruber, R. Selective retina therapy (SRT): a review on methods, techniques, preclinical and first clinical results. Bull Soc Beige Ophtalmol, 51-69 (2006).
29. Paulus, Y. M. et al. Selective retinal therapy with microsecond exposures using a continuous line scanning laser. Retina 31, 380-388 (2011).
30. Sramek, C. et al. Non-damaging retinal phototherapy: dynamic range of heat shock protein expression. Invest Ophthalmol Vis Sci 52, 1780-1787 (2010).

While some aspects of the present invention have been shown and described herein, it will be obvious to those skilled in the art that such aspects are provided by way of example only. Numerous variations, changes, and substitutions will now occur to those skilled in the art without departing from the invention. It should be understood that various alternatives to the aspects of the invention described herein may be employed in practicing the invention. It is intended that the following claims define the scope of the invention and that methods and structures within the scope of these claims and their equivalents be covered thereby.

### EXAMPLES

It should be understood that the following examples used only to clarify the present invention, but not to limit this invention.

It must be explained, if not specified, that the percentage of following examples are all weight percent content wt %.

### Example 1

### Selectivity of RPE Destruction

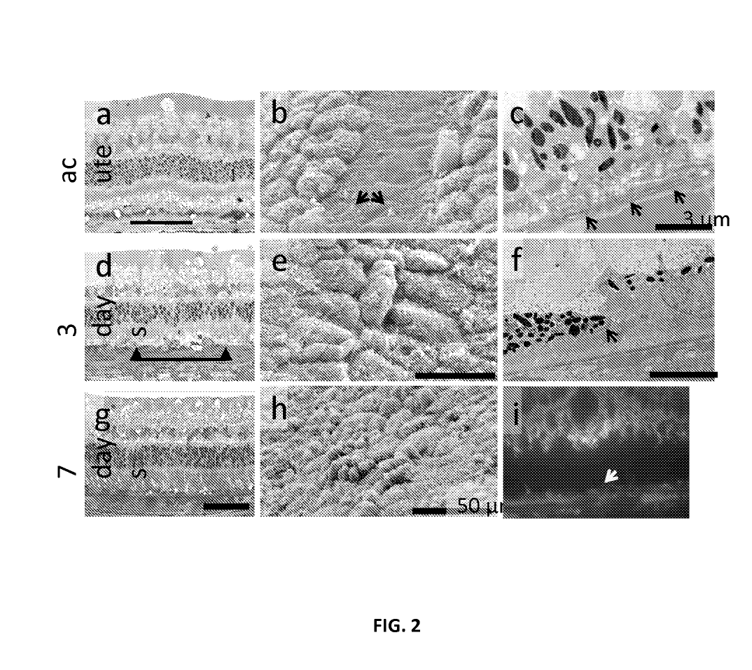

Pigmented rabbits are transfected using a rAAV2 encoding Green Fluorescent Protein (GFP). Selectivity of RPE destruction was tested with line scanning laser in vivo using fluorescence angiography and spectral domain optical coherence tomography (SD-OCT), and confirmed with light microscopy, transmission (TEM) and scanning electron microscopy (SEM). A typical example of selective destruction of RPE cells produced by scanning laser in a rabbit eye without damage to photoreceptors and Bruch's membrane is shown in FIG. 2. Collapsed RPE cells underneath the intact photoreceptors can be seen in the histological section (FIG. 2a). SEM image shows absence of the RPE cells along the line of laser scanning one day after treatment and evidence of cellular migration by foot processes on the surrounding cells (FIG. 2b). TEM demonstrates RPE disruption with an intact Bruch's membrane underneath. Continuity of the RPE layer is reestablished in three days (FIG. 2d and e), including restoration of the tight junctions between RPE cells, as observed in TEM (FIG. 2f). After 7 days appearance of the RPE cells in the damaged area is further normalized, and normal anatomy of the overlying outer segments of photoreceptors is restored (FIG. 2g-h).

Bromodeoxyuridine (BrdU) immunohistochemistry assay of cell proliferation identified positive signal in approximately 30% of the RPE cells in the treated area, suggesting that the healing process involves both proliferation of RPE cells as well as cell migration and stretching (FIG. 2i).

### Example 2

### Control Expression of GFP in Retina

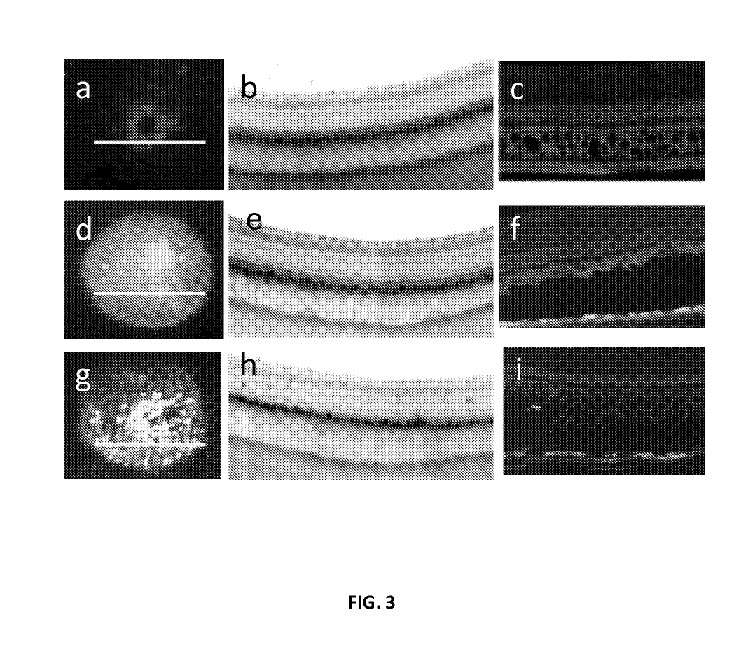

Each eye received three subretinal injections: one with balanced salt solution (BSS), and two containing an rAAV vector encoding GFP. One bleb was left untreated for control and another was treated by SRT 3 weeks after surgery with a line scanning pattern density of 50% (i.e. laser lines were spaced one line width apart). GFP fluorescence was mapped to quantify the level of GFP expression in transfected retina over time. Possible neurosensory retina damage was monitored in-vivo using high resolution SD-OCT (FIG. 3a-b, d-e, g-h).

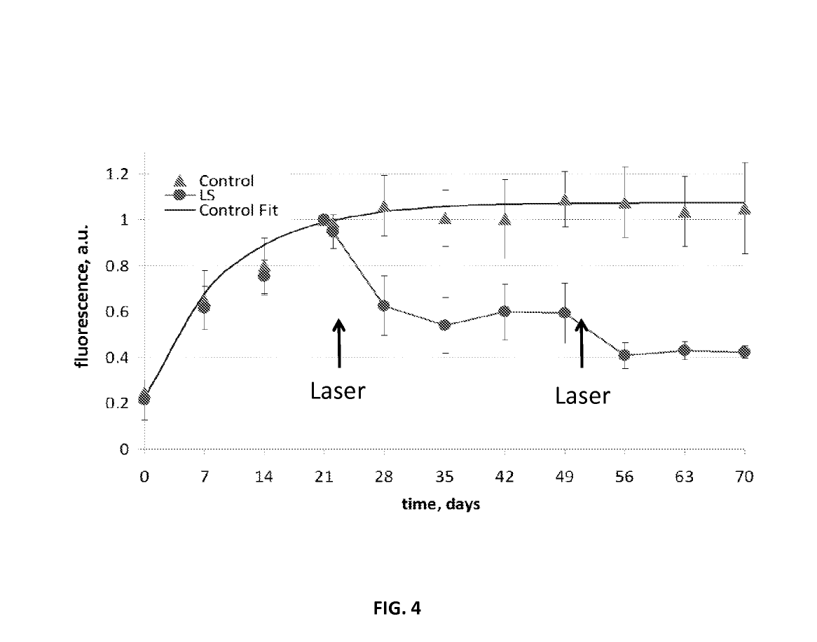

The time course of GFP expression and the effect of SRT on fluorescent signal was plotted in FIG. 4. Both transfected blebs reached maximum fluorescence level at 3 weeks. There was no significant increase in fluorescence signal in the BSS-injected bleb. Fluorescence in the control bleb (transfected, but not treated with laser) remained stable throughout the follow up period. The laser-treated bleb showed a 40% signal decrease within a week, which remained stable for four weeks, when the same area was treated second time, and resulting in additional reduction by half the initial amount: 20%.

In vivo imaging and ultra-structural analysis with TEM and SEM confirmed that after the second treatment there was a similar pattern of damage and following restoration of the RPE monolayer, and no damage was observed to the neurosensory retina or Bruch's membrane. Fluorescence microscopy showed normal autofluorescence in the BSS-injected bleb (FIG. 3c), intense RPE fluorescence homogenously distributed over the control transfected bleb (FIG. 3f) and interposed areas of intense fluorescence and decreased signal in the SRT treated bleb (FIG. 3i). No significant expression of GFP was detected in the neurosensory retina.

### Example 3

### Up-regulation of GFP in Retina by Application of Laser

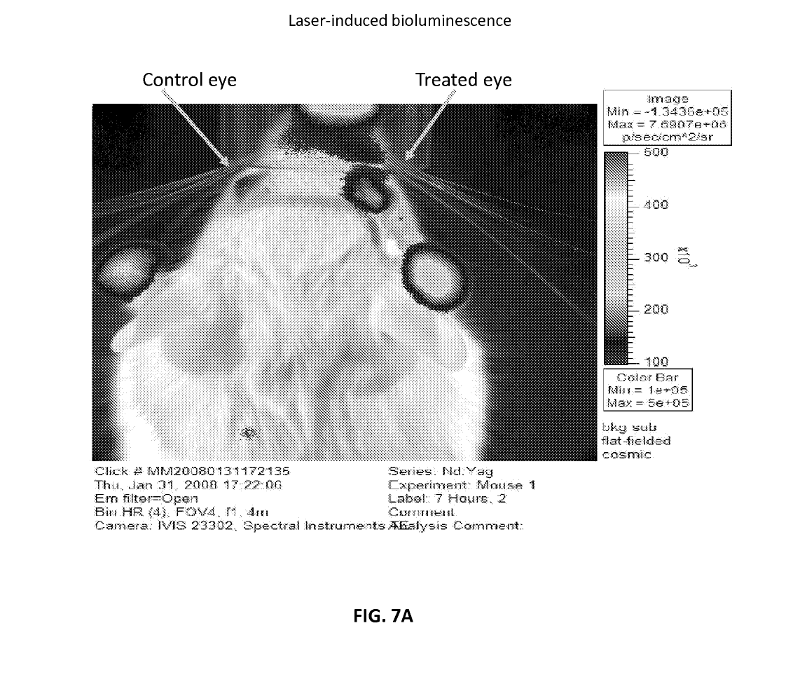

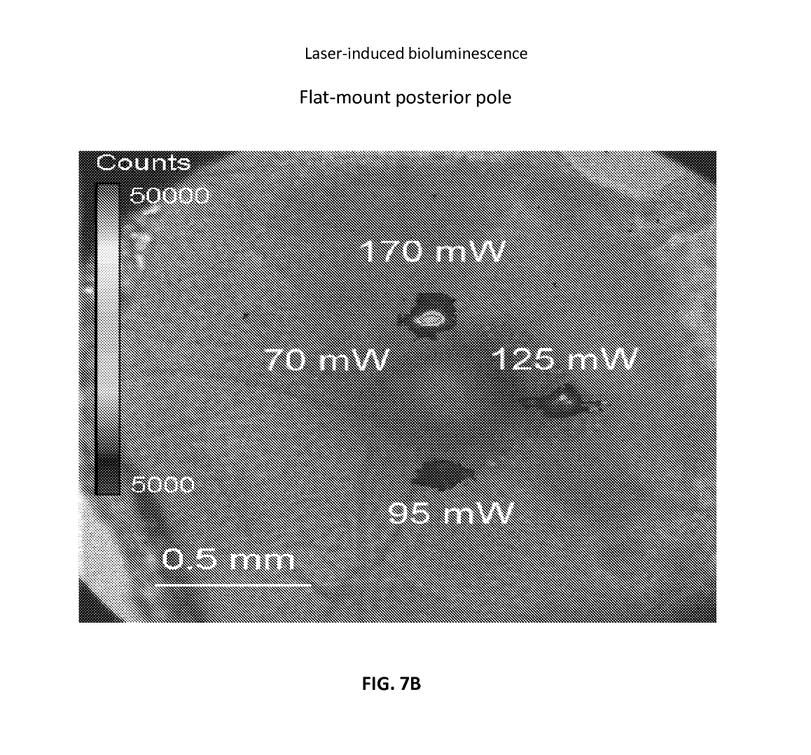

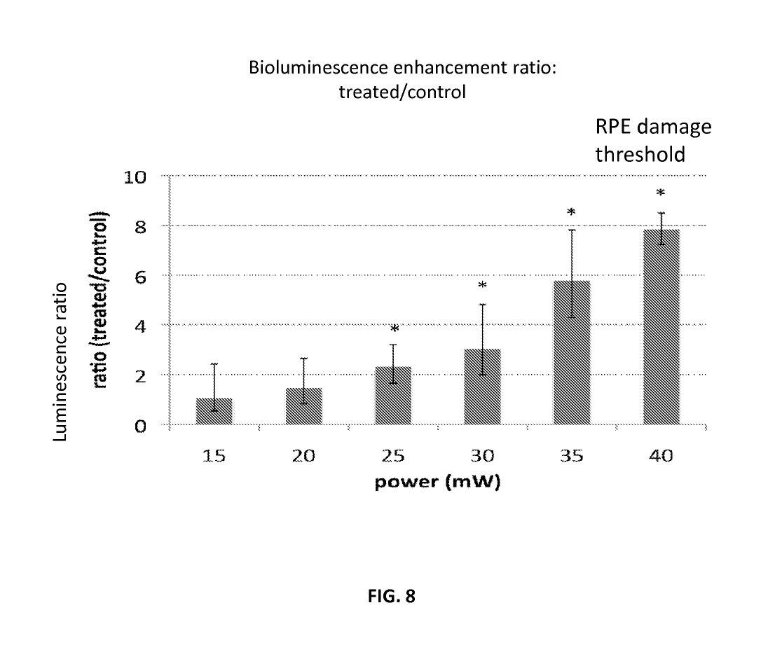

To visualize the expression of heat shock protein, a transgenic mouse expressing luciferase under the control of a heat-shock sensitive promoter (HSP70) were used to visualize laser-induced bioluminescence indicating genetic up-regulation. FIG. 7 illustrates enhanced expression of the heat-shock protein HSP70 visualized by bioluminescent light emission from the treated area in an albino mouse. FIG. 8 illustrates increase of the bioluminescence with laser dose below the RPE damage threshold (40 mW) of a pigmented mouse.

1. A method for modulating gene expression in a subject, comprising;
(a) expressing a transgene in a plurality of cells in a subject, wherein said plurality of cells comprises a target cell, wherein said target cell comprises;
(i) a polynucleotide encoding said transgene; and
(ii) a thermo generator capable of generating heat upon application of an electromagnetic radiation; and
(b) applying said electromagnetic radiation to said target cell to destroy said target cell, thereby modulating expression of said transgene in said subject.

2. The method of claim 1, wherein said target cell is a pigmented cell and said thermo generator comprises a chromophore or pigment.

3. The method of claim 2, wherein said pigmented cell is selected from the group consisting of: retinal pigment epithelium, iris epithelium, trabecular meshwork epithelium, and a cell in the pigmented choroid.

4.-10. (canceled)

11. The method of claim 1, wherein said electromagnetic radiation is produced by a laser.

12. The method of claim 11, wherein said laser has a beam width of less than 1 mm.

13. The method of claim 11, wherein said laser has a power of about 500 mW to 5,000 mW.

14. The method of claim 1, wherein said electromagnetic radiation is applied by one or more pulses.

15. The method of claim 14, wherein the duration of said pulse of electromagnetic radiation is less than 100 μs.

16. The method of claim 1, wherein said electromagnetic radiation is applied by scanning a beam of electromagnetic radiation.

17. (canceled)

18. The method of claim 16, wherein the scanning beam has a dwell time less than 100 μs.

19. A method for modulating gene expression in a target cell by electromagnetic radiation, comprising;
(a) expressing a transgene in a target cell, wherein said target cell comprises:
(i) a polynucleotide encoding heat-responsive promoter that controls expression of said transgene; and
(ii) a thermo generator that is capable of generating heat upon application of an electromagnetic radiation; and
(b) applying said electromagnetic radiation to said target cell to increase the expression of said transgene.

20. The method of claim 19, wherein said target cell is a pigmented cell and said thermo generator comprise a chromophore or pigment.

21. The method of claim 19, wherein said pigmented cell is selected from the group consisting of: retinal pigment epithelium, iris epithelium, trabecular meshwork epithelium, and a cell in the pigmented choroid.

22.-28. (canceled)

29. The method of claim 19, wherein said electromagnetic radiation is a laser.

30. The method of claim 29, wherein said laser has a beam width of less than 1 mm.

31. The method of claim 29, wherein said laser has a power of about 5 mW to 500 mW.

32. The method of claim 19, wherein said electromagnetic radiation is applied by one or more pulses.

33. The method of claim 32, wherein the duration of applying said pulses of electromagnetic radiation is less than 100 μs.

34. The method of claim 19, wherein said electromagnetic radiation is applied by scanning a beam of electromagnetic radiation.

35. The method of claim 34, wherein the scanning beam has a dwell time less than 100 μs.
